(19) United States
(12) Patent Application Publication
(10) Pub. No.: US 2020/0279654 A1
(43) Pub. Date: Sep. 3, 2020

(54) SOFTWARE, HEALTH STATUS DETERMINATION DEVICE AND HEALTH STATUS DETERMINATION METHOD

(71) Applicant: FUYO KAIHATSU CO.,LTD., Fukuoka-shi (JP)

(72) Inventor: Shunsuke MAETA, Fukuoka-shi (JP)

(21) Appl. No.: 16/633,143

(22) PCT Filed: Nov. 15, 2018
(86) PCT No.: PCT/JP2018/042365

§ 371 Date: Jan. 22, 2020

(30) Foreign Application Priority Data
Nov. 15, 2017 (JP) 2017-220418

(51) Int. Cl. G16H 50/30; A61B 5/0205; A61B 5/00; G16H 10/60; G16H 50/20; G16H 50/50

(52) U.S. Cl. CPC G16H 50/30; A61B 5/02055; A61B 5/742; A61B 5/021; G16H 50/20; G16H 50/50; G16H 10/60

(57) ABSTRACT

A health status determination device which reflects vital signs or daily conditions to contribute to a subject's health care, or the provision of medical care, includes a processing unit that executes one or more programs to cause the health status determination device to function as an information input means, an information recording means, a reference calculation means, a scoring processing means, and a determination processing means to perform transmission and reception of information, recording of information, scoring based on the contents of vital information, configuring of scoring conditions (scoring reference information), abnormality determination in score value information, and configuring of abnormality determination reference, notification of the determination result, creation or display of display information.

### TECHNICAL FIELD

The present disclosure relates to software, a health status determination device and a health status determination method. Specifically, the present disclosure relates to software, a health status determination device and a health status determination method which reflect vital signs or daily conditions in consideration of the individual differences of the subjects, enable the subjects to grasp the intra-individual variation with high accuracy, and contribute to the subject's health care or the provision of medical care for the characteristics of each individual.

### BACKGROUND ART

Recently, the importance of “personalized medicine” in the medical field is increasing. The personalized medicine refers to “performing medical care for the personality of each person”, called tailor-made medicine.

Until now, medical care has been performed based on an accident centered on a disease, and the main purpose is to search for the cause of the disease or to develop a treatment method for the disease. On the other hand, the status of a disease varies greatly from person to person, and it has been known that it is not always right to apply the same treatment even if the disease is the same.

However, in conventional medical treatment, individual differences in therapeutic effects are considered unknown without observing the treatment and the effects thereof, and thus, it is difficult to perform an optimal treatment plan for each individual.

Here, it is important to grasp “biomarkers” different for each individual in realizing personalized medicine. In general, a biomarker is an indicator of a status of a particular disease or a status of life, and a research group at the National Institutes of Health defined biomarkers in 1988 as “characteristics that are objectively measured and evaluated as indicators of pharmacological responses to common biological processes, pathological processes, or therapeutic interventions.” Also, in the past, biomarkers mainly meant physiological indicators such as blood pressure, heart rate, and the like.

Also, in the medical field, a scoring method called an early warning score (hereinafter referred to as “EWS”) is used as a technique for rapidly evaluating the degree of a subject's illness.

The EWS is a technique for calculating a score according to a measurement result or an evaluation result of vital signs, based on the measurement of the subject's six major signs: respiratory rate (rpm), oxygen saturation (SpO2) (%), body temperature (° C.), blood pressure (mmHg), heart rate (bpm), and consciousness level (AVPU response: A: alert (normal), V: voice (responsive to voice), P: pain (responsive to pain), U: unresponsive), and determining the degree of disease by the total score of the score (refer, e.g., non-patent literature 1).

This EWS is based on the principle that the clinical degradation can be seen via changes in multiple physiological measurements and large changes within a single variable. In addition, when calculating a score from the measurement values of each vital sign, numerical values determined based on the result of the measurement value of a group (a plurality of subjects) is adopted.

For example, as shown in Table 1, in the case of body temperature, “36.0˜37.9° C.” is configured as a center range of the measurement value, and it becomes a score of zero (0) point in this range. In addition, for the upper and lower values, the measurement values of body temperature and the scores of the scoring are configured so that if it is “35.0˜35.91° C.” or “38.0˜38.91° C.”, the score becomes 1 point, if it is “34.0˜34.91° C.” or “a value exceeding 38.9° C.”, the score becomes 2 points, and if it is “less than 34° C.”, the score becomes 3 points. In addition, other vital signs are configured as shown in Table 1.

TABLE 1

| Score | 3 | 2 | 1 | 0 | 1 | 2 | 3 |
|---|---|---|---|---|---|---|---|
| Respiratory | >35 | 31~35 | 21~30 | 9~20 | | | <7 |
| rate | | | | | | | |
| (Respiratory | | | | | | | |
| rate/min) | | | | | | | |
| SpO2 (%) | <85 | 85~89 | 90~92 | >92 | | | |
| Temperature | | >38.9 | 38~38.9 | 36~37.9 | 35~35.9 | 34~34.9 | <34 |
| (° C.) | | | | | | | |
| Systolic | | >199 | | 100~199 | 80~99 | 70~79 | <70 |
| blood | | | | | | | |
| pressure | | | | | | | |
| (mmHg) | | | | | | | |
| Heart rate | >129 | 110~129 | 100~109 | 50~99 | 40~49 | 30~39 | <30 |
| (bpm) | | | | | | | |
| AVPU | | | | Alert | Voice | Pain | Uncon- |
| | | | | | | | sciousness |

Here, in the content shown in Table 1, “normal range” and “abnormal range” configured from the measurement values of vital signs of a group are adopted as the references. The range configured here can be changed in consideration of the area, age, etc., but basically, the range used as a reference is determined based on the measurement values of the vital signs obtained from the majority of the number of people. The configuration of the reference is the same for respiratory rate, oxygen saturation, blood pressure, and heart rate.

Further, in the EWS, scores may be assigned to other parameters such as urine output, oxygen dose flow rate, and pain score as parameters other than the six major vital signs.

### CITATION LIST

### Patent Documents

Non-patent document 1: “Early warning score”, [online], Wikipedia, the free encyclopedia, searched on Oct. 16, 2017, Internet<URL:https://en.wikipedia.org/wiki/Early_warning_score>

### SUMMARY OF INVENTION

### Technical Problem

However, the conventional mechanism for scoring based on the measurement values of the subject's vital signs, including the EWS described in Non-Patent Document 1, and detecting abnormality, based on the information of the result, was not a detection in consideration of variation in the subject's individual.

In the Non-Patent Document 1, as described above, the “normal range” and the “abnormal range” configured based on the measurement value of the vital signs of a group become the references. For this reason, it is difficult to say that the detection takes into account the intra-individual variation of the subject.

In other words, the reference configured from the measurement values of the vital signs of a group cannot correspond to the individual characteristics in the vital signs. For example, in young people and elderly people, the body temperatures in a calm state, or the temperature fluctuations in one day are very different. Moreover, the value of the vital sign varies greatly between subjects, depending on the presence or absence of a disease state such as hypertension.

That is, in consideration of the age of the subject or the presence or absence of a disease state, the “normal range” or “abnormal range” configured from the measurement values of the vital signs of a group may not be an appropriate reference.

In the personalized medicine field, which is expected to expand in the future, biomarkers suitable for the purpose of diagnosis, prognosis, pharmacodynamics, and monitoring are indispensable. The inventors have so far conducted research focusing on biomarkers aimed at “onset of aging” and “diagnosis”.

Among them, the inventors have found the possibility of using “vital signs” of body temperature, pulse, and blood pressure (pulse pressure), which are the most basic information about human life, as biomarkers, rather than biomarkers that perform analysis at the current genetic level. The body temperature, pulse rate, and blood pressure (pulse pressure), together with the respiratory rate, are called “classical vital signs” in the medical field, and these four have become the basic and important vital signs.

The reason why these classical vital signs can be used as biomarkers is because the measurements of body temperature, blood pressure (pulse pressure), pulse rate, and respiratory rate have “individual variations” that differ from person to person. In other words, it was thought that the method of change of vital signs differs depending on the subject, and the technique that contributed to the health management or diagnosis of the subject could be developed by appropriately grasping and interpreting the method of change.

In addition, the inventors have found, through the previous research, that if at least 30 pieces of measurement data of the classical vital signs of human beings have been acquired from the same individual, the measurement values basically follow a normal distribution. In addition, the normal distribution of the classical vital signs includes variations in the person's own individual health.

However, there are papers and reports of “inter-individual variation” that observe many people's standard deviations of vital signs in medical statistics, but none of the “variations within an individual” of the same subjects are addressed.

In addition, in the prior art, there are no results of analyzing that the measurement value of the classical vital sign for the same individual follows the normal distribution or techniques for using this for abnormality determination. As a matter of course, there was no technique using the fact that the measurement value of the classical vital signs follows the normal distribution as a reference for the scoring technique used for the above-described abnormality determination.

The present disclosure was made in view of the above points, and the present disclosure relates to software, a health status determination device and a health status determination method which reflect vital signs or daily conditions in consideration of the individual differences of the subjects, enable the subjects to grasp the intra-individual variation with high accuracy, and contribute to the subject's health care or the provision of medical care for the personality of each individual.

### Solution to Problem

In order to achieve the above purpose, software of the present disclosure is software for scoring vital information which is information related to obtained vital signs to determine a health status of a subject, based on the obtained score result information, the software making an information processing device function as a means including: an information input means for receiving an input of vital information including at least one measurement value obtained from the same individual and selected from classical vital signs following a normal distribution and acquisition date and time; an information recording means for recording the input vital information and information on the acquisition date and time; a reference calculation means for calculating a mean μ and a standard deviation σ of all or part of a plurality of the recorded vital information; a scoring processing means for scoring an input predetermined vital information to calculate score result information which is a value of a score, based on a predetermined scoring condition, wherein the predetermined scoring condition is configured based on a normal distribution having at least the mean μ as a peak value; and a score determination means for determining whether the score result information is an abnormal value, based on a predetermined score determination condition, wherein the vital information includes a value determined as an abnormal value by the score determination means, the score determination means performs the next determination, the vital information of at least 30 pieces of the measurement data reflects the variation within the individual unique to the individual, the normal distribution is made with the vital information of at least 30 pieces of measurement data, the vital information includes at least one measurement value selected from body temperature, blood pressure, pulse rate and pulse pressure which are the classical vital signs, the scoring condition, for at least one measurement value selected from body temperature, blood pressure, pulse rate and pulse pressure, has a value of Equation 1 below as a lower limit and a value of Equation 2 below as an upper limit, the Equation 1 and Equation 2 being represented using the mean μ, the standard deviation σ, n and m that are numbers greater than 0 (zero), and has at least one of the lower limit and the upper limit as a reference:

μ−nσ Equation 1; and

μ+mσ Equation 2.

In addition, the software in the specification is a program related to the operation of a computer. The program is also made up of a numbered sequence of instructions suitable for processing by a computer.

In addition, in the present disclosure, the “vital information of 30 pieces of measurement data” is data of vital information measured broadly every second, and includes different lengths of time like data of vital information measured, for example, every minute, several minutes, every hour, every day, every month. In addition, “the vital information of 30 pieces of measurement data” includes not only data regularly acquired at regular intervals, such as every 1 second or every 1 minute, but also irregularly acquired data. For example, at least 30 pieces of data obtained at irregular intervals (acquisitions such as 1 second, 3 seconds, 6 seconds, 7 seconds, 9 seconds, etc.) may be adopted, instead of being acquired at regular intervals for one minute (60 seconds). In addition, for example, at least 30 pieces of data acquired irregularly for 30 minutes, at least 30 pieces of data acquired irregularly for 1 hour, at least 30 pieces of data acquired irregularly for several hours, at least 30 pieces of data acquired irregularly for 1 day, at least 30 pieces of data acquired irregularly for several days, at least 30 pieces of data acquired irregularly for 1 week, at least 30 pieces of data acquired irregularly for several weeks, at least 30 pieces of data acquired irregularly for 1 month, etc. may be adopted as the “vital information of 30 pieces of measurement data”. Further, at least 30 pieces of data can be randomly extracted from the accumulated vital information regardless of the regular intervals or irregular intervals, and can be adopted as the “vital information of 30 pieces of measurement data”. As will be described later, if at least 30 pieces of measurement data are acquired regardless of the length of time or the regularity of the measurement interval, a normal distribution reflecting the variation in the individual of the subject can be obtained.

Here, the information input means receives the input of the vital information acquired from the same individual and the information recording means records the input vital information, so that the vital information of the same individual can be accumulated. In addition, the same individual as used herein refers to the determination subject which is determined as to whether the value of the acquired vital sign is an abnormal value.

In addition, the term “individual” as used herein means a single organism (human or animal). In addition, the present disclosure is single software and includes an aspect of recording the vital information of a single identical individual, and an aspect of recording a plurality of pieces of vital information of the same individual for each same individual. The same individual refers to the same person, for human being, for example.

In addition, the term “the vital information acquired from the same individual” as used herein means that the individual can be distinguished at the stage of input by the information input means. For example, it is also possible to distinguish an individual by different formats of input, such as an aspect in which one subject inputs personal vital information, or an aspect in which a specific personal input screen is displayed when the subject deals with information of a plurality of subjects, and inputs the vital information, or the like.

In addition, the information input means receives input of vital information including at least one measurement value which is obtained from the same individual and selected from classical vital signs following a normal distribution, and records the input vital information in the information recording means, so that it is possible to accumulate information on measurement values of classical vital signs of the same individual. The measurement values of the classical vital signs obtained from the same individual follow the normal distribution, and by accumulating the information of the measurement values, it is possible to configure a reference based on the normal distribution.

In addition, the information input means receives the input of the vital information acquired from the same object and the information of the acquisition date, and records the input vital information and the information of the acquisition date and time in the information recording means, so that the vital information of the same individual is accumulated with the information on the acquisition date and time. That is, the plurality of pieces of vital information of the same individual can be handled in association with the information on the acquisition date and time. In addition, when comparing with other vital information, it is possible to confirm the situation of displacement and the displacement amount between the vital information to be compared. The information on the acquisition date and time herein includes an aspect in which the inputter inputs information on an acquisition date and time when inputting vital information in the information input means, or an aspect in which the time at which the vital information is input is automatically input in the information input means. The information on the acquisition date and time includes the date and time when the vital signs were measured or the date and time when the vital signs were evaluated (e.g., consciousness level).

In addition, the reference calculation means calculates the mean μ of all or a part of the plurality of recorded vital information, so that the information of the mean value of the vital information reflecting the variation in the individual of the same individual can be used. In addition, the mean μ as used herein means the value obtained by dividing “the total of the measurement value of each vital sign” by “the number of data of the measurement value of vital sign”. In addition, the term “the mean μ of a plurality of recorded vital information” as used herein includes not only that calculated from the entire data of the recorded vital information but also that calculated from a part of the entire data. Furthermore, the vital information that is the basis for calculating the mean μ may be calculated from continuous data, for example, measured continuously such as every second, every minute, every hour, every day, as well as data extracted at intervals of seconds, minutes, hours, and dates.

Further, the reference calculation means calculates the standard deviation σ of all or part of a plurality of recorded vital information so that it is possible to use information of the standard deviation of the vital information reflecting variation in the individual of the same individual. In addition, the standard deviation σ as used herein is “square mean of the deviation” of the vital information of a predetermined condition. More specifically, the “deviation” is a value obtained by subtracting the “mean value of measurement values of the vital signs under a predetermined condition” from “the measurement values of each vital signs” of the vital information under a predetermined condition. The “standard deviation σ of a plurality of recorded vital information” includes not only that calculated from the entire data of the recorded vital information but also that calculated from a part of the entire data. Further, the vital information that is the basis for calculating the standard deviation σ may be calculated from not only continuous data such as data measured continuously such as every second, every minute, every hour, or every day, but also data extracted at intervals of seconds, minutes, hours, and dates.

Further, the scoring processing means scores the input predetermined vital information to calculate the score result information which is the value of the score, based on a predetermined scoring condition, thereby converting the input vital information into score result information (score) according to the content thereof.

In addition, a predetermined scoring condition is configured based on a normal distribution having at least the mean μ as a peak value, so that when the measurement value of the classical vital signs obtained from the same individual is input as the vital information, the score result information according to the content can be obtained by the reference configured based on the normal distribution having the mean μ as the peak value. In addition, the reference configured based on the normal distribution with the mean μ as the peak value at this time is a reference which reflects the variation of the individual of the same individual, and it becomes possible to score the vital information within the same individual while reflecting the variation within the individual. The term “input predetermined vital information” as used herein means vital information that is the basis for scoring. In addition, the term “a predetermined scoring condition configured based on the normal distribution having at least the mean μ as a peak value” as used herein includes both that configured by including input predetermined vital information, that is, predetermined vital information to be scored and that configured from the previous vital information without including the predetermined vital information to be scored. In addition, the input predetermined vital information may be recently input vital information. In addition, the input predetermined vital information may be one or a plurality of vital information among the previously input vital information.

Further, the score determination means determines whether the score result information is an abnormal value based on a predetermined score determination condition, so that it is possible to determine whether the score result information obtained from the contents of the vital information obtained from the same individual is an abnormal value. In addition, in the determination based on the predetermined score determination conditions herein, there may be an aspect of determining whether the score result information obtained from one vital sign is an abnormal value or an aspect of determining the total points of a plurality of score result information, and furthermore, an aspect of determining the combination of two or more pieces of score result information.

Further, the vital information includes a value determined as an abnormal value by the score determination means, the score determination means performs the next determination, the vital information of the at least 30 pieces of measurement data reflects the intra-individual variation unique to the individual, and the normal distribution is made up of vital information of at least 30 pieces of measurement data, so that the personalized medicine can be realized by utilizing the vital information as a biomarker. In particular, the deterioration of the status of the subject in the chronic phase can be detected early. That is, the vital information can be used as a “biomarker” different for each individual.

This biomarker is generally defined as an indicator of a particular disease status or status of life, and is defined as “a characteristic that is objectively measured and evaluated as an indicator of a pharmacological response to a normal biological process, pathological process, or therapeutic intervention.” That is, the “biomarker for detecting the deterioration of a health condition”, which is the target of the present disclosure, is an indicator unique to the subject reflecting various states of the subject, including a state in which the subject is calm, a state in which any abnormality has occurred in the condition, and a state before an abnormal condition occurs.

In the present disclosure, as described above, the vital information including the value determined as an abnormal value by the score determination means is used, and this enables the subject to grasp the manner of the change in vital signs, including the state in which any abnormality has occurred in the condition and the state before the abnormal condition occurs. In addition, the manner of changing the vital sign reflects the width of change which varies for each subject, which is normally distributed. The change in vital signs includes fluctuations in different individuals in each person, and it becomes possible to carry out health care or diagnosis of the subject and to realize personalized medicine by using and interpreting the vital signs including the variation in the individual as a biomarker. Further, the value determined as an abnormal value by the score determination means is included and the score determination means performs the next determination, so that it is possible to continuously perform the determination of the subject, based on the vital sign including the variation in the individual.

In addition, in the present disclosure, by using vital information including a value determined as an abnormal value, it is possible to appropriately grasp variation in other individuals in each subject to utilize it for early detection of a condition in which an abnormal condition occurs. In addition, the present disclosure can be used for so-called self-management in relation to self-management and prevention of the condition at the stage before the abnormal condition occurs.

These effects are particularly meaningful for the elderly in a chronic phase, in which the condition is relatively stable but is difficult to cure, because early detection is possible before abnormalities occur remarkably in the condition after reflecting the characteristics of the individual. In the elderly, the progress of the disease is often low, and it is difficult to grasp the change in condition and the deterioration of the condition as compared with general adults. However, deterioration can be detected early in accordance with the characteristics of the subject by using the vital signs including the variation within the individual of the subject as a biomarker.

In addition, the vital information of the at least 30 pieces of measurement data reflects the variation within individual unique to the individual, and the normal distribution is made up of the vital information of at least 30 pieces of measurement data, so that it is possible to sufficiently grasp the abnormal value of the vital sign in the subject of interest by the score determination means.

Here, the technical significance of adopting “30 pieces of measurement data” in the present disclosure will be described. More specifically, it will be explained that if at least 30 pieces of measurement data are acquired for the vital signs regardless of the length of time or the regularity of the measurement interval, the normal distribution reflecting the variation in the individual of the subject can be obtained.

The inventors have confirmed that, based on previous studies, if at least 30 pieces of measurement data can be acquired for the vital data obtained from the same individual, the measurement data is normally distributed to reflect the variation within the individual for each subject.

For example, as shown in FIGS. 15 to 22, when the pulse was measured under each condition, if 30 pieces of measurement data were prepared, it is possible to obtain normal distribution curves different for each subject based on the measured data. FIGS. 15, 17, 19 and 21 are the results of the pulses obtained from the same subject (herein, referred to as A), and FIGS. 16, 18, 20 and 22 are result of a pulse obtained from other identical subjects (herein, referred to as B). In addition, although 30 pieces of measurement data are shown in FIGS. 15 to 22 and the circle on a curve corresponds to a piece of measurement data, since there exists a plurality of overlapping data centered on a mean value, 30 circles does not appear in the figures.

More specifically, FIGS. 15 and 16 are graphs based on the results obtained by measuring pulses every minute and acquiring measurement data of 30 pulses. Results were obtained in which all showed the shape of the normal distribution with the mean value as the vertex. Moreover, in the cases of A and B, the mean values used as the vertex differ, and also the values located at both ends of a curve (minimum value and maximum value) differ. Thus, it is clear that a normal distribution of individual subjects is obtained. This point is also confirmed in the same tendency in FIGS. 17 to 22.

In addition, FIGS. 17 and 18 are graphs based on the results obtained by measuring pulses every 7 minutes and obtaining measurement data for 30 pulses. Thus, even when the time interval to measure was changed, the shape of the normal distribution which made the mean value of each subject the vertex was obtained.

In addition, FIGS. 19 and 20 are graphs based on the results of acquiring measurement data of 30 pieces of pulses at irregular times during the day. Furthermore, FIG. 21 is a graph based on the result of obtaining the measurement data of 30 pieces of pulses at an irregular time for 30 hours, and FIG. 22 is a graph based on the result of obtaining the measurement data of 30 pieces of pulses at an irregular time for 30 days. As shown here, it was confirmed that if 30 pieces of measurement data were acquired even if the data was not regularly acquired at regular intervals, the data took the form of a normal distribution with the mean value of each subject as the vertex.

In addition, for example, as shown in FIGS. 23 and 24, when the 30 pieces of measurement data for body temperature were also prepared, the normal distribution curve different for each subject was obtained based on the measurement data. FIGS. 23 and 24 are graphs based on the results obtained by measuring body temperature every two minutes to acquire 30 pieces of measurement data of body temperature. In addition, in FIGS. 23 and 24, the subjects who were measured for the body temperature were different. Thus, even in the case of body temperature, it was confirmed that if the 30 pieces of measurement data were acquired, the data took the shape of the normal distribution with the mean value of each subject as the vertex.

The point that the normal distribution of each individual was obtained from 30 pieces of measurement data was confirmed not only for pulse and body temperature but also for blood pressure (systolic blood pressure and diastolic blood pressure), pulse pressure, and respiratory rate.

Therefore, the inventors have found that a normal distribution reflecting the variation within the individual of the subject can be obtained by obtaining at least 30 pieces of measurement data for the vital signs regardless of the length of time or the regularity of the measurement interval and that the possibility of using the 30 pieces of measurement data as a biomarker, and reached the present disclosure.

That is, regardless of the length of time or the regularity of the measurement interval, at least 30 pieces of vital information can be obtained to acquire a normal distribution so that the physiological parameters (the vital signs) of the subject can be observed and the value of the vital sign constituting the period can be checked. In other words, when 30 pieces of vital information are used, it is possible to sufficiently grasp the abnormal value of the vital sign in the individual of the subject, and the present disclosure is focused on this point.

Further, in order to achieve the purpose, software of the present disclosure is software for scoring vital information which is information related to obtained vital sign to determine a health status of a subject, based on the obtained score result information, and is software for making an information processing device function as a means including: an information input means for receiving an input of vital information including at least one measurement value obtained from the same individual and selected from classical vital signs following a normal distribution and acquisition date and time; an information recording means for recording the input vital information and information on the acquisition data and time; a reference calculation means for calculating a mean μ and a standard deviation σ of all or a part of a plurality of recorded vital information; a scoring processing means for scoring the input vital information to calculate score result information which is a value of a score, based on an input predetermined scoring condition, wherein the predetermined scoring condition is configured based on a normal distribution having at least the mean μ as a peak value; a score determination means for determining whether the score result information is an abnormal value, based on a predetermined score determination condition; and a vital determination means for determining whether the input predetermined vital information is an abnormal value, based on a predetermined numerical range for vital determination, configured based on at least one selected from the mean μ and the standard deviation σ, the predetermined numerical range for vital determination being configured based on a normal distribution with at least the mean μ as a peak value, wherein the vital information includes a value determined as an abnormal value by the score determination means, the score determination means performs the next determination, the vital information of the at least 30 pieces of measurement data reflects the variation within the individual unique to the individual, the normal distribution is made up of the vital information of at least 30 pieces of measurement data, the vital information includes at least one measurement value selected from body temperature, blood pressure, pulse rate and pulse pressure which are the classic vital signs, the vital determination means uses a value of Equation 1 below as a lower limit and a value of Equation 2 below as an upper limit, the Equation 1 and Equation 2 being represented using the mean μ, the standard deviation σ, n and m that are numbers greater than 0 (zero), and determines whether the input predetermined vital information is an abnormal value, based on at least one of the lower limit and the upper limit:

μ−nσ Equation 1; and

μ+mσ Equation 2.

Here, the input predetermined vital information is determined as to whether it is an abnormal value, based on the predetermined range for vital determination configured based on at least one selected from the mean μ and the standard deviation σ, and the predetermined vital determination numerical range has a reference in which the intra-individual variation of the same individual is reflected on the value of the vital information itself by the vital determination means configured based on a normal distribution having at least the mean μ as the peak value and whether the vital information is an abnormal value can be determined. In other words, it is possible to determine whether the value is an abnormal value with respect to the measurement value of the individual vital information, separately from the determination of whether the abnormal value is related to the score result information. In addition, the predetermined numerical range for vital determination which is a determination reference is configured using a mean value or a standard deviation calculated from the vital information accumulated for the same individual and therefore is unique to the same individual, and it is possible to determine whether it is abnormal with a reference in which the mean value of the vital information or the dispersion from the mean value is reflected. The term “input predetermined vital information” as used herein means vital information that is the object of determination. In addition, the term “predetermined numerical range for vital determination” referred to herein includes a numerical range configured to include input predetermined vital information, i.e., predetermined vital information to be determined, and a numerical range configured from previous vital information without including predetermined vital information to be determined. In addition, the term “predetermined numerical range for vital determination” includes both an aspect in which the numerical value to be determined is determined to be “abnormal” when a reference value, for example, the upper limit value is configured and the numerical value is equal to or higher than the upper limit value, and an aspect in which the numerical value is determined to be “abnormal” when the numerical value to be determined exceeds the upper limit. The input predetermined vital information may be recently input vital information. In addition, the input predetermined vital information may be one or a plurality of vital information among previously input vital information.

In addition, the vital information includes a value determined as an abnormal value by the vital determination means, the vital information of the at least 30 pieces of measurement data reflects the variation within the individual unique to the individual, and the normal distribution is made up of vital information of at least 30 pieces of measurement data. Thus, the vital information can be utilized as a biomarker to realize the personalized medicine, and in particular, the deterioration of the condition of a subject in a chronic period can be detected early. That is, the vital information can be used as a “biomarker” different for each individual.

In addition, the intra-individual variation unique to the individual is reflected in the vital information of the at least 30 pieces of measurement data, and the normal distribution is made up of the vital information of the at least 30 pieces of measurement data, so that it is possible to sufficiently grasp the abnormal value of the vital sign in the object of interest by the vital determination means.

In addition, when the vital information has at least one measurement value selected from body temperature, blood pressure, pulse rate and pulse pressure, which are classical vital signs, score result information can be obtained for body temperature, blood pressure, pulse rate and pulse pressure measured from the same individual to determine whether it is an abnormal value.

In addition, in a case where the scoring condition is a predetermined numerical range configured based on a normal distribution having a mean μ as a peak value for at least one measurement value selected from body temperature, blood pressure, pulse rate and pulse pressure, the score result information according to the contents can be obtained by the reference configured based on the normal distribution with the mean μ as the peak value when the measurement values of body temperature, blood pressure, pulse rate and pulse pressure obtained from the same individual are input as vital information. In addition, the reference configured based on the normal distribution having the mean μ as the peak value at this time is a reference in which the variation in the individual of the same individual is reflected, and the vital information of the same individual can be scored in the form reflecting the variation in the individual. In addition, the term “predetermined numerical range” used herein includes both an aspect in which, when a reference value, for example, a certain value is configured, a numerical value of targets for scoring becomes two points above a certain value and one point when it is less than the certain value, and the numerical value of targets for scoring becomes two points when exceeding the certain value and one point below the certain value.

In addition, when the vital information has a measurement value of oxygen saturation, score result information can be obtained for the oxygen saturation measured from the same individual to determine whether it is an abnormal value.

In addition, when the scoring condition is a predetermined numerical range configured previously for the measurement value of oxygen saturation, if the measurement value of oxygen saturation acquired from the same individual was input as vital information, the score result information corresponding to the content can be obtained based on a predetermined numerical range configured in advance. In addition, the “predetermined numerical range” used herein can adopt the numerical range configured from the measurement values of the vital signs of a group. In addition, the term “predetermined numerical range” used herein includes both an aspect in which, when a reference value, for example, a certain value is configured, a numerical value of targets for scoring becomes two points above a certain value and one point when it is less than the certain value, and the numerical value of targets for scoring becomes two points when exceeding the certain value and one point below the certain value.

In addition, when the vital information has a measurement value of the respiratory rate, it is possible to obtain score result information on the respiratory rate measured from the same individual to determine whether it is an abnormal value.

In addition, when the scoring condition is a predetermined numerical range configured based on a mode or a mean μ by calculating the mode or the mean μ of all or a part of the measurement values of the recorded plurality of respiratory rate with respect to the measurement value of the respiratory rate, if the measurement value of the respiratory rate acquired from the same individual is input as vital information, the score result information according to the content can be obtained by the reference configured based on the mode or the mean value. In addition, the reference configured based on the mode or mean value at this time reflects the characteristics of the respiratory rate of the same individual, and the vital information of the same individual can be scored with this reference. In addition, the term “the reference configured based on a mode value or a mean value” used herein includes both an aspect in which, when a reference value, for example, a certain value is configured, a numerical value of targets for scoring becomes two points above a certain value and one point when it is less than the certain value, and the numerical value of targets for scoring becomes two points when exceeding the certain value and one point below the certain value.

In addition, when the vital information has a consciousness level evaluation result obtained by observing a consciousness level, score result information can be obtained with respect to the consciousness level evaluation result obtained from the same individual, and it can be determined as to whether it is an abnormal value.

In addition, when the scoring condition is a predetermined observation state indicating the degree of consciousness level with respect to the consciousness level evaluation result, the consciousness level evaluation result obtained from the same individual is applied to the contents of the predetermined observation state and the score according to the content. The result information may then be obtained. The content of the predetermined observation state is, for example, the content of the AVPU response used for the evaluation of the consciousness level or the content indicating the state of confusion.

In addition, the scoring condition, for at least one measurement value selected from body temperature, blood pressure, pulse rate and pulse pressure, has a value of Equation 1 below as a lower limit and a value of Equation 2 below as an upper limit, the Equation 1 and Equation 2 being represented using the mean μ, the standard deviation σ, n and m that are numbers greater than 0 (zero), and has at least one of the lower limit and the upper limit as a reference, it is possible to score based on the numerical value which is separated by the value of no in the negative direction from the mean μ as the lower limit, and the numerical value which is separated by the value mσ from the mean μ as an upper limit:

μ−nσ Equation 1; and

μ+mσ Equation 2.

In other words, the value obtained by subtracting no from the mean μ is configured as the lower limit and the value of the mean μ added to mσ is configured as an upper limit value, based on at least one of them, and score result information can be obtained with respect to measurement values of body temperature, blood pressure, pulse rate and pulse pressure measured from the same individual. In addition, the values of n and m may be any number greater than 0 as described above and the values of n and m can be appropriately configured in consideration of various conditions such as the strictness of the standard, the type of vital signs, and the history of the subject. In addition, since it is “at least one of a lower limit and an upper limit,” it includes not only the aspect which configures only a lower limit or only an upper limit as a reference but the aspect which adopts both a lower limit and an upper limit as a reference. In addition, the value of Equation 1 and Equation 2 here is a numerical range configured based on the normal distribution which configures the mean μ as the peak value.

In addition, when the information input means receives input of information on the disease status, information on the history, the observation information on the condition, and the information on the care record of the same individual, and the information recording means records information on the disease status, information on the past history, observation information on the condition, and information on the care record of the input same individual, detailed information on the health status of the same individual can be accumulated.

In addition, when the thermal table processing means creates a thermal table, based on the vital information recorded on the information recording means, the information on the disease status, the information on the history, the observation information on the condition and the information vital information on the care record, detailed information about the health status of the same individual can be listed in the thermal table along with the vital information.

In addition, when the reference calculation means calculates the mean μ and the standard deviation σ from at least two vital information of a predetermined period recorded in the information recording means, it is possible to calculate the mean μ and standard deviation σ from the partial information without using the entire data of the plurality of recorded vital information.

Further, when the score determination means determines the score result information as an abnormal value, when the abnormality is determined by dividing the abnormality into at least two stages, the treatment after the determination of the score result information can be diversified. For example, even if the status indicates abnormality, if the numerical value of the score result information is small, it is notified as “caution”, and if the numerical value of the score result information is large, it is notified as “warning”, so that not all abnormalities are treated uniformly. As a result, when the determination is performed, it is possible to efficiently deal with the post-judgment treatment such as whether a doctor's check is immediately necessary.

In addition, when the vital determination means configures a value of Equation 1 below represented using the mean μ, standard deviation σ, n and m that are numbers greater than zero (0) as a lower limit, and a value of Equation 2 below as the upper limit, and determines whether the input predetermined vital information is an abnormal value, based on at least one of the lower limit value and the upper limit value, it is possible to determine the presence or absence of abnormality with respect to the value of the vital information, based on the value separated by the value of no in the negative direction from the mean μ as a lower limit and the value separated by the value of mσ from the mean μ as an upper limit:

μ−nσ Equation 1; and

μ+mσ Equation 2.

That is, the value obtained by subtracting no from the mean μ is configured as a lower limit value and a value obtained by adding mσ to the mean μ is configured as an upper limit value, and at least one of them may be used to determine whether the vital information measured from the same individual is an abnormal value. In addition, the values of n and m may be any number greater than as mentioned above, and these values of n and m can be configured suitably considering various conditions such as the strictness of the standard, the type of the vital sign, and the history of the subject. In addition, since the values correspond to at least one of the “lower limit value” and the “upper limit value”, not only an aspect in which only a lower limit value or only an upper limit value is configured as a reference, but also an aspect in which both a lower limit value and an upper limit value are adopted as references are included. In addition, the value of Equation 1 and Equation 2 here is a numerical range configured based on the normal distribution having the mean μ as a peak value.

Further, in order to achieve the purpose, a health status determination device of the present disclosure is a health status determination device for scoring vital information which is information related to obtained vital signs to determine a health status of a subject, based on the obtained score result information, and is configured to include: an information input means for receiving an input of vital information including at least one measurement value obtained from the same individual and selected from classical vital signs following a normal distribution and acquisition date and time; an information recording means for recording the inputted vital information and information on the acquisition data and time; a reference calculation means for calculating a mean μ and a standard deviation σ of all or a part of multiple recorded vital information; a scoring processing means for scoring the inputted vital information to calculate score result information which is a value of a score, based on an input predetermined scoring condition, the predetermined scoring condition being configured based on a normal distribution having at least the mean μ as a peak value; a score determination means for determining whether the score result information is an abnormal value, based on a predetermined score determination condition; and a display means capable of displaying the determination result determined by the score determination means, wherein the vital information includes a value determined as an abnormal value by the score determination means, the vital information of at least 30 pieces of the measurement data reflects the variation in the individual unique to the individual, and the normal distribution is made up of the vital information of at least 30 pieces of measurement data.

Here, the information input means may receive an input of vital information including at least one measurement value obtained from the same individual and selected from the classical vital signs following a normal distribution, and the input vital information is recorded in the information recording means, so that information of measurement values of the classical vital signs of the same individual can be accumulated. The measurement values of the classical vital signs obtained from the same individual follow the normal distribution, the measurement values are accumulated, so that a reference based on the normal distribution can be configured.

In addition, the predetermined scoring condition is configured based on a normal distribution with at least the mean μ as a peak value, so that when the measurement value of the classical vital sign obtained from the same individual is input as the vital information, the score result information according to the content can be obtained by the reference configured based on the normal distribution with the mean μ as a peak value. In addition, the reference configured based on the normal distribution with the mean μ as the peak value at this time is the reference which reflects the fluctuation of the individual within the same individual, and it is possible to score the vital information of the same individual in the state in which the fluctuation in the individual is reflected. The term “input predetermined vital information” as used herein means vital information that is to be scored. The term “predetermined scoring condition configured based on the normal distribution with at least the mean μ as the peak value” as used herein includes both that configures to include the predetermined vital information input, that is, the predetermined vital information to be scored and that configured from the previous vital information without including the predetermined vital information to be scored. In addition, the input predetermined vital information may be recently input vital information. In addition, the input predetermined vital information may be one or a plurality of vital information among previously input vital information.

Moreover, the determination result determined by the score determination means can be displayed by the display means and be confirmed.

Further, in order to achieve the purpose, a health status determination device of the present disclosure is a health status determination device for scoring vital information which is information related to obtained vital signs to determine a health status of a subject, based on the obtained score result information, and is configured to include: an information input means for receiving an input of vital information including at least one measurement value obtained from the same individual and selected from classical vital signs following a normal distribution and acquisition date and time; an information recording means for recording the inputted vital information and information on the acquisition data and time; a reference calculation means for calculating a mean μ and a standard deviation σ of all or a part of multiple recorded vital information; a scoring processing means for scoring the inputted vital information to calculate score result information which is a value of a score, based on an input predetermined scoring condition, wherein the predetermined scoring condition is configured based on a normal distribution having at least the mean μ as a peak value; a score determination means for determining whether the score result information is an abnormal value, based on a predetermined score determination condition; a vital determination means for determining whether input predetermined vital information is an abnormal value, based on a predetermined numerical range for vital determination configured based on at least one selected from the mean μ and the standard deviation σ, wherein the predetermined numerical range for vital determination is configured based on a normal distribution with at least the mean μ as a peak value; and a display means capable of displaying the determination result determined by the score determination means, wherein the vital information includes a value determined as an abnormal value by the vital determination means, the vital information of at least 30 pieces of the measurement data reflects the variation in the individual unique to the individual, and the normal distribution is made up of the vital information of at least 30 pieces of measurement data.

Here, based on the predetermined range for the vital value determination configured based on at least one selected from the mean μ and the standard deviation σ, the input predetermine vital information is determined as to whether it is an abnormal value, and the predetermined numerical range for vital determination has a reference in which the variation in the individual of the same individual is reflected, and it is possible to determine whether it is an abnormal value for the vital information of the same individual, for the value itself of the vital information, by the vital determination means configured based on a normal distribution with at least the mean μ as a peak value. That is, it is possible to determine whether the value is an abnormal value with respect to the value of the individual vital information measured separately from the determination of whether it is an abnormal value with respect to the score result information. In addition, the predetermined range for vital determination as the determination reference is configured using the mean value or the standard deviation calculated from the vital information accumulated for the same individual, and therefore is unique to the same individual, and is determined from the mean value or mean value of the vital information. In addition, it is possible to determine whether it is abnormal with a reference in which the mean value of the vital information or the dispersion from the mean value is reflected.

Further, in order to achieve the purpose, a health status determination method of the present disclosure is a method executed by a computer and a health status determination method for scoring vital information which is information related to obtained vital signs to determine a health status of a subject, based on the obtained score result information, and is configured to include: an information recording process for receiving and recording an input of vital information including at least one measurement value obtained from the same individual and selected from classical vital signs following a normal distribution; a reference calculating process for calculating a mean μ and a standard deviation σ of all or a part of multiple recorded vital information; a scoring processing process for scoring the inputted vital information to calculate score result information which is a value of a score, based on an input predetermined scoring condition, wherein the predetermined scoring condition is configured based on a normal distribution having at least the mean μ as a peak value; and a score determining process (*determining process) for determining whether the score result information is an abnormal value, based on a predetermined score determination condition (* predetermined determination condition), wherein the vital information includes a value determined as an abnormal value by the score determination process, the vital information of at least 30 pieces of the measurement data reflects the variation in the individual unique to the individual, and the normal distribution is made up of the vital information of at least 30 pieces of measurement data.

Here, in the information recording process, by receiving and recording an input of vital information including at least one measurement value obtained from the same individual and selected from classical vital signs following a normal distribution, information of measurement values of the classical vital signs of the same individual can be accumulated. The measurement values of the classical vital signs obtained from the same individual follow the normal distribution, the measurement values are accumulated, so that a reference based on the normal distribution can be configured.

In addition, in the reference calculating process, by calculating the mean μ of all or a part of multiple recorded vital information, it is possible to use information of a mean of the vital information reflecting the fluctuation of the individual within the same individual. In addition, the mean μ used herein means a value obtained by dividing the “total of measurement values of each vital sign” by the “number of data of measurement values of the vital signs”. Incidentally, the term “mean μ of the plurality of recorded vital information” includes not only that calculated from the entire data of the recorded vital information but also that calculated from a part of the entire data. Further, the vital information on which the mean μ is calculated may be calculated from continuous data, for example, data continuously measured at every second, every minute, every hour, every day, as well as from data extracted at intervals of seconds, minutes, hours, and dates.

In addition, in the reference calculating process, by calculating the standard deviation σ of all or part of the plurality of recorded vital information, it is possible to use the information of the standard deviation of the vital information reflecting the variation in the individual of the same individual. In addition, the standard deviation σ used herein is the “square mean of the deviation” of the vital information of a predetermined condition. More specifically, “deviation” is a value obtained by subtracting the “mean value of the measurement values of the vital signs under predetermined conditions” from the “measurement values of each vital signs” of the vital information under predetermined conditions. Incidentally, the term “standard deviation σ of the plurality of recorded vital information” used herein includes not only that calculated from the entire data of the recorded vital information but also that calculated from a part of the entire data. Furthermore, the vital information on which the standard deviation σ is calculated may be calculated from not only continuous data such as data measured continuously such as every second, every minute, every hour, or every day, but also from data extracted at intervals of seconds, minutes, hours, and dates.

In addition, in the scoring process, based on a predetermined scoring condition, the inputted vital information can be scored to calculate score result information which is a value of the score, so that the inputted vital information can be converted into score result information (score) according to the content.

In addition, a predetermined scoring condition is configured based on a normal distribution with at least the mean μ as the peak value, so that when the measurement value of the classical vital sign obtained from the same individual is input as the vital information, the score result information according to the content can be obtained by the reference configured based on the normal distribution with the mean μ as the peak value. In addition, the reference configured based on the normal distribution with the mean μ as the peak value at this time is a reference which reflects the variation of the individual within the same individual, and it becomes possible to score the vital information of the same individual while reflecting the variation within the individual. The term “input predetermined vital information” as used herein means vital information that is to be scored. In addition, the “predetermined scoring condition configured based on the normal distribution with at least the mean μ as a peak value” as used herein includes both that configured by including input predetermined vital information, that is, predetermined vital information to be scored and that configured from the previous vital information without including the predetermined vital information to be scored. In addition, the input predetermined vital information may be recently inputted vital information. In addition, the input predetermined vital information may be one or a plurality of vital information among previously inputted vital information.

Further, in the scoring determination process, by determining whether the score result information is an abnormal value based on a predetermined score determination condition, it is possible to determine whether the score result information obtained from the contents of the vital information obtained from the same individual is an abnormal value. In addition, in the determination based on the predetermined score determination conditions herein, there may be an aspect of determining whether the score result information obtained from one vital sign is an abnormal value or an aspect of determining the total points of several score result information, and furthermore, an aspect of determining the combination of two or more score result information.

Further, the vital information includes a value determined as an abnormal value in the score determining process, the vital information of the at least 30 pieces of measurement data reflects the intra-individual variation unique to the individual, the normal distribution is made up of vital information of at least 30 pieces of measurement data, so that personalized medicine can be realized by utilizing the vital information as a biomarker, and in particular, the deterioration of the status of the subject in the chronic phase can be detected early. That is, the vital information can be used as a “biomarker” different for each individual.

Further, the vital information of the at least 30 pieces of measurement data reflects the intra-individual variation unique to the individual, and the normal distribution is made up of vital information of at least 30 pieces of measurement data, so that it becomes possible to sufficiently grasp the abnormal value of the vital sign in the subject of interest by the score determination process.

Further, in order to achieve the purpose, a health status determination method of the present disclosure is method executed by a computer, and is a health status determination method for scoring vital information which is information related to obtained vital signs to determine a health status of a subject, based on the obtained score result information, the method being configured to include: an information recording process for receiving and recording an input of vital information including at least one measurement value obtained from the same individual and selected from classical vital signs following a normal distribution; a reference calculating process for calculating a mean μ and a standard deviation σ of all or a part of multiple recorded vital information; a scoring processing process for scoring the inputted vital information to calculate score result information which is a value of a score, based on an input predetermined scoring condition, wherein the predetermined scoring condition is configured based on a normal distribution having at least the mean μ as a peak value; a score determining process (*determining process) for determining whether the score result information is an abnormal value, based on a predetermined score determination condition (* predetermined determination condition); and a vital determining process for determining whether the predetermined vital information input is an abnormal value, based on a predetermined numerical range for vital determination configured based on at least one selected from the mean μ and the standard deviation σ, the predetermined numerical range for vital determination being configured based on a normal distribution with at least the mean μ as a peak value, wherein the vital information includes a value determined as an abnormal value by the vital determination process, the vital information of at least 30 pieces of the measurement data reflects the variation in the individual unique to the individual, and the normal distribution is made up of the vital information of at least 30 pieces of measurement data.

Here, by the vital determining process for determining whether the predetermined vital information input is an abnormal value, based on a predetermined numerical range for vital determination configured based on at least one selected from the mean μ and the standard deviation σ and the predetermined numerical range for vital determination is configured based on a normal distribution with at least the mean μ as a peak value, it is possible to determine whether it is an abnormal value for the vital information of the same individual, with the reference in which the variation in the individual of the same individual is reflected, for the value itself of the vital information. That is, it is possible to determine whether the value is an abnormal value with respect to the value of the measured individual vital information, apart from determining whether the score result information is abnormal. Further, since the predetermined range for vital determination as the determination reference is configured using the mean value or the standard deviation calculated from the vital information accumulated for the same individual, it is possible to determine whether it is abnormal with a reference that is unique to the same individual and reflects the mean value of the vital information or the dispersion from the mean value.

Further, the vital information includes a value determined as an abnormal value in the vital determining process, the vital information of the at least 30 pieces of measurement data reflects the intra-individual variation unique to the individual, the normal distribution is made up of vital information of at least 30 pieces of measurement data, so that personalized medicine can be realized by utilizing the vital information as a biomarker, and in particular, the deterioration of the status of the subject in the chronic phase can be detected early. That is, the vital information can be used as a “biomarker” different for each individual.

Further, the vital information of the at least 30 pieces of measurement data reflects the intra-individual variation unique to the individual, and the normal distribution is made up of vital information of at least 30 pieces of measurement data, so that it becomes possible to sufficiently grasp the abnormal value of the vital sign in the subject of interest by the vital determination process.

### Advantageous Effects of Invention

The software, a health status determination device and a health status determination method according to the present disclosure are capable of grasping the subject's individual variation with high accuracy by reflecting the vital signs and daily conditions in consideration of the individual's individual differences, and can contribute to the health care of the subject and the provision of medical care for each individual person.

### BRIEF DESCRIPTION OF DRAWINGS

FIG. 1 is a view showing a schematic configuration of a tablet terminal incorporating software to which the present disclosure is applied (1 first system configuration);

FIG. 2 is a schematic diagram showing a second system configuration with software to which the present disclosure is applied;

FIG. 3 is a schematic diagram showing a third system configuration with software to which the present disclosure is applied;

FIG. 4 is a block diagram showing the structure of an operation unit, an information transmission/reception unit, and an information recording unit;

FIG. 5 is a schematic diagram showing an example of the configuration of a calculation period of the vital mean value and the vital standard deviation;

FIG. 6 is a schematic diagram showing an example of extraction of vital information;

FIG. 7A is a schematic diagram showing an example of a device used when functioning the software to which the present disclosure is applied;

FIG. 7B is a schematic diagram showing another example of the device;

FIG. 8 is a schematic diagram illustrating an example of an input screen of a value of a vital sign;

FIG. 9 is a schematic diagram illustrating another example of an input screen of a value of a vital sign;

FIG. 10A is a graph of a normal distribution curve created based on vital information of a plurality of subjects, and FIG. 10B is a graph of a normal distribution curve created based on vital information of the same subject;

FIG. 11 is a schematic view showing an example of a columnar table;

FIG. 12 is a schematic diagram illustrating an example of an image showing a result of scoring in an electronic chart;

FIG. 13 is a schematic diagram showing an example of an image showing a result of scoring by application software used in a smartphone terminal;

FIG. 14 is a flowchart showing the flow of information processing from input of vital information to abnormality determination in score value information and display of result information;

FIG. 15 is a normal distribution curve based on the result of having acquired the measurement data of 30 pulses by measuring a pulse every minute;

FIG. 16 is a normal distribution curve based on the result of having acquired the measurement data of 30 pulses by measuring a pulse every minute;

FIG. 17 is a normal distribution curve based on the result of having acquired the measurement data of 30 pulses by measuring a pulse every 7 minutes;

FIG. 18 is a normal distribution curve based on the result of having acquired the measurement data of 30 pulses by measuring a pulse every 7 minutes;

FIG. 19 is a normal distribution curve based on the results of obtaining measurement data of 30 minutes of pulses at irregular times during the day;

FIG. 20 is a normal distribution curve based on the results of acquiring measurement data of 30 minutes of pulses at irregular times during the day;

FIG. 21 is a normal distribution curve based on the results of acquiring measurement data of 30 minutes of pulses at irregular times for 30 hours;

FIG. 22 is a normal distribution curve based on the results of obtaining measurement data of 30 minutes of pulses at irregular times during 30 days;

FIG. 23 is a normal distribution curve based on the result of measuring body temperature every two minutes and obtaining measurement data of thirty body temperatures; and

FIG. 24 is a normal distribution curve based on the result of measuring body temperature every two minutes and obtaining measurement data of thirty body temperatures.

### DETAILED DESCRIPTION OF THE EMBODIMENTS OF THE INVENTION

Hereinafter, embodiments of the present disclosure will be described with reference to the drawings and an understanding of the present disclosure will be provided.

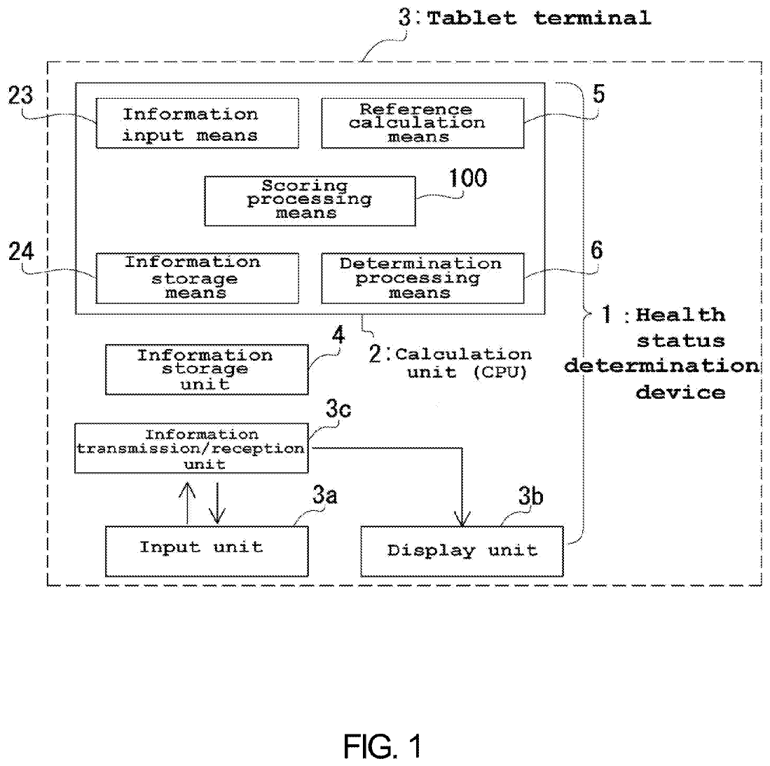

FIG. 1 is a view showing a schematic configuration of a tablet terminal incorporating software to which the present disclosure is applied. In addition, the structure shown below is an example of the present disclosure, and the content of the present disclosure is not limited thereto.

[1. About Whole Device Configuration]

The software to which the present disclosure is applied can be introduced into a general-purpose information processing device, and provides each information processing function necessary for implementing the present disclosure to the mounted information processing device. As a result, in the tablet terminal 3, it is possible to input the vital information of the subject to perform scoring according to the content thereof, and determine whether the obtained score result information (hereinafter, referred to as “score value information”) is an abnormal value.

Further, the information processing device includes a calculating unit such as a CPU, a storage unit such as a RAM or a ROM, an input unit such as a display screen such as a liquid crystal screen or a keyboard, a communication unit for controlling communication with the Internet, and the like. For example, the information processing device is a general purpose personal computer, a tablet terminal, a smart phone, or the like. In addition, as the information processing device, for example, various health care devices, or a medical system or a nursing system installed in a hospital, a facility, or the like may also be covered, and software to which the present disclosure is applied may be mounted and used.

The software to which the present disclosure is applied is downloaded and mounted on the tablet terminal 3 as application software, and a tablet terminal having a scoring function of vital information and a function of determining a score value is used as the health status determination device 1.

In addition, hereinafter, the user of the health status determination device 1, that is, the person whose health status is determined, is called a “subject.”

As shown in FIG. 1, the health status determination device 1 (tablet terminal 3) has a calculation unit 2. The calculation unit 2 is a processing unit which performs each information processing function which the health status determination device 1 has. In other words, in the software to which the present disclosure is applied, the calculation unit 2 of the tablet terminal 3 functions as an information input means 23, an information recording means 24, a reference calculation means 5, a scoring processing means 100, a determination processing means 6, or the like. The processing function of each of these means allows the transmission and reception of information, recording of information, scoring based on the contents of vital information, configuring of scoring conditions (scoring reference information), abnormality determination in score value information, abnormality determination regarding score values, configuring of abnormality determination reference in score value, notification of determination results regarding score value, determination of abnormalities in the values of vital signs, configuring of abnormal judgment reference regarding values of vital signs, notification of judgment results regarding values of vital signs, creation or display of indication information, or the like. In addition, the tablet terminal 3 can access an external server, a terminal, and the like through the Internet, and can transmit and receive information between an external server, a terminal, and the like. The information recording means 24, the reference calculation means 5, the scoring processing means 100, and the determination processing means 6 are respectively examples of an “information recording means”, a “reference calculation means”, a “scoring processing means”, a “score determination means” and a “vital determination means.”

The tablet terminal 3 has an information recording unit 4, an information transmission and reception unit 3c, an input unit 3a, and a display unit 3b.

The information transmission and reception unit 3c is a part that is responsible for transmitting and receiving information between the calculating unit 2, the information recording unit 4, the input unit 3a, the display unit 3b, and the like. In addition, the information transmission and reception unit 3c may be configured to enable the transmission and reception of information between the tablet terminal 3 and the external terminal.

Here, hereinafter, each piece of information handled by the software to which the present disclosure is applied does not necessarily need to be recorded in the information recording unit 4 of the tablet terminal 3. For example, an aspect of the present disclosure may include transmitting and recording various information to an external server or an external terminal through the information transmitting and receiving unit 3c of the tablet terminal 3, and receiving necessary information from the external server or the like when performing a judgment or the like.

More specifically, it is not necessary for all of the main components of the health status determination device 1 to be downloaded to the tablet terminal 3. For example, the tablet terminal 3 may only display information such as determination result information or a thermal table, and recording and determination processing of various information may be performed by an external server.

The software to which the present disclosure is applied may have a plurality of variations in configuration of the system. Examples of some variations are described below.

(First System Configuration)

In a schematic configuration of the tablet terminal 3 shown in FIG. 1, the software to which the present disclosure is applied is introduced into the terminal so that the terminal itself can input and record vital information, display score values, determine score values, display determination results of the score values, configure scoring conditions, configure the determination calculation reference of the score values, determine the value of the vital signs, display the determination result of the values of the vital signs, and configure the determination calculation reference of the values of the vital signs. That is, the device itself can perform the functions of the present disclosure. The schematic configuration shown in FIG. 1 shows the use of the software to which the present disclosure is applied in a device of the “stand alone type” not connected to the Internet environment. The software of the present disclosure can be introduced into an information processing device that is not connected to the Internet environment, for example, various healthcare devices, medical systems/nursing systems such as hospitals, and used as a dedicated device. In addition, since the tablet terminal 3 is exemplified of an information processing device, the connection with the Internet environment is possible. However, the configuration shown in FIG. 1 enables the determination of health status only by the internal function of the tablet terminal 3.

(Second System Configuration)

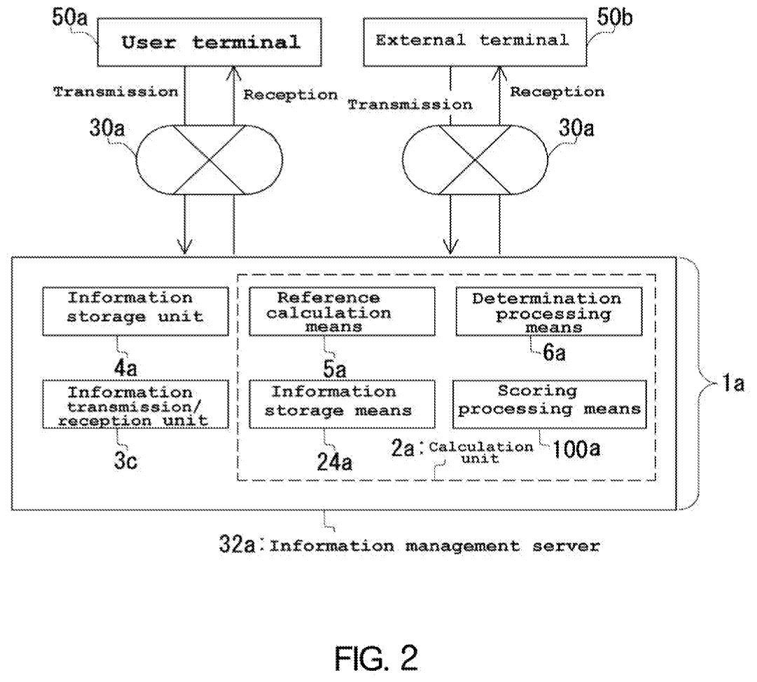

In FIG. 2, a structure which gives an external server the function of the software 1a to which the present disclosure is applied can also be adopted as a second system structure. Here, a user terminal 50a or an external terminal 50b can access the information management server 32a via the Internet 30a. The information management server 32a is an external server provided in a cloud form, for example, and can use the function of the software 1a to which the present disclosure is applied on the information management server 32a.

The information management server 2a has an information recording unit 4a, an information transmitting and receiving unit 3c, and a calculating unit 2a. Moreover, the calculating unit 2a has a reference calculation means 5a, an information recording means 24a, a scoring processing means 100a, and a determination processing means 6a. The input of the vital information is performed through the user terminal 50a or the external terminal 50b, and information input from each terminal is transmitted to the information management server 32a to record information and determine score value on the information management server 32a side. The determination result of the score value and the recorded information can be transmitted to the user terminal 50a or the external terminal 50b, and can be confirmed by each terminal. Thus, the system structure which gives the function of the software 1a on an external server can also be adopted.

(Third System Configuration)

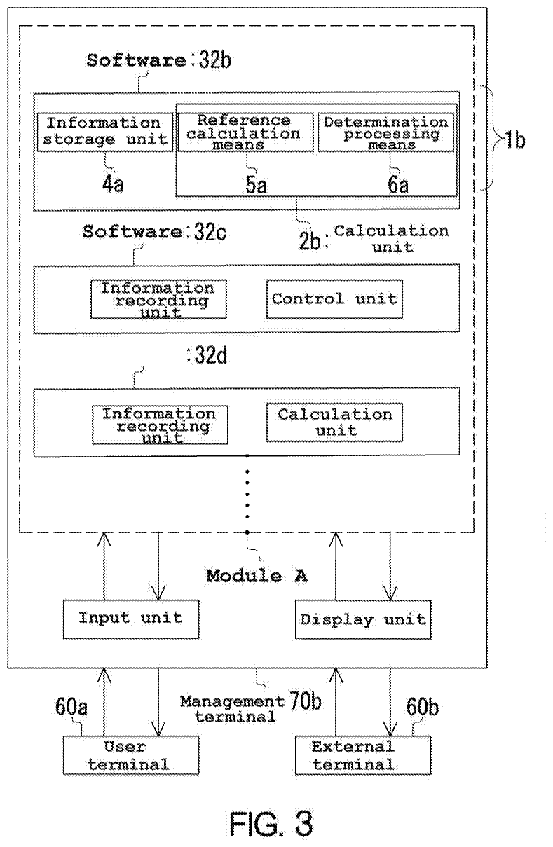

In FIG. 3, a configuration of a management terminal 70b including a module A having a plurality of software 32c and 32d and the like in addition to the function of software 32b to which the present disclosure is applied, as a third system configuration is shown. The software 32b to which the present disclosure is applied constitutes the module A together with other software for enabling the management terminal 70b to perform various functions different from itself. In other words, it is possible to mount the software 32b on the module A of the management terminal 70b in which the plurality of software 32c and 32d, or the like has been introduced in advance, and make it function. For example, the software to which the present disclosure is applied can also be mounted in the module included in a management terminal of a medical system such as an electronic chart.

In this third system configuration, vital information is input to the management terminal 70b to perform scoring and determination of the score value, and the result information can be confirmed on the management terminal 70b. In addition, the user terminal 60a or the external terminal 60b are connected to the management terminal 70b so that the vital information is input from the user terminal 60a or the external terminal 60b and transmitted to the management terminal 70b, the management terminal 70b performs scoring and determination of the score values, and the result information may be received and confirmed by the user terminal 60a or the external terminal 60b. As such, the software to which the present disclosure is applied can also adopt a configuration that functions as part of a module composed of a plurality of software.

As described above, the configuration on the system of the software (or health status determination device) to which the present disclosure is applied has a plurality of variations. In addition, although the above description has been mainly focused on three examples, the configuration of the software (or health status determination device) to which the present disclosure is applied is not limited thereto. For example, an information recording unit may be provided in the user terminal, and the reference calculation means, the scoring processing means, and the determination processing means may be given to an external server to divide the location of necessary functions into the terminal and the server. That is, if the vital information of the subject is recorded, the determination reference (scoring condition, the numerical range for the vital determination) reflecting the variation in the individual are configured, and the determination of the state of health is possible, various configurations can be adopted.

Detailed configuration will be described below using the usage aspect of the tablet terminal 3 shown in FIG. 1.

[2. Information Recording Unit]

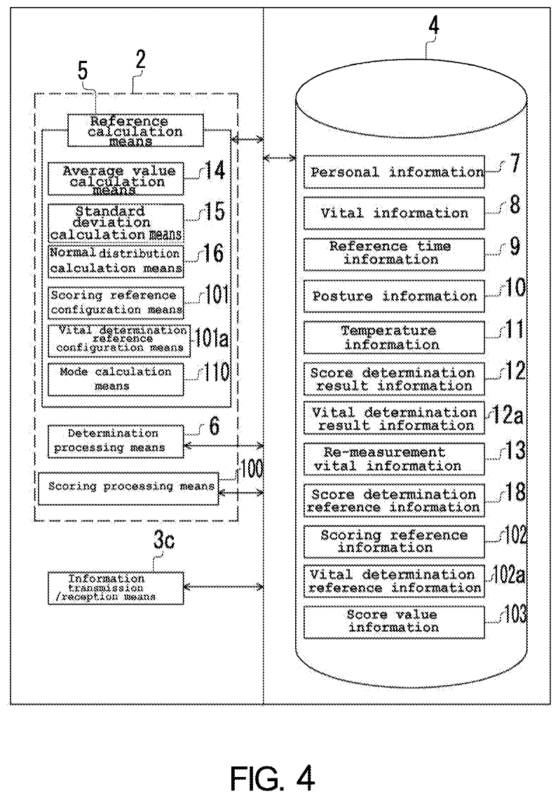

As shown in FIG. 4, various information is recorded in the information recording unit 4.

The information recording unit 4 is a part that stores the vital information including the personal information of the subject and the value of the vital signs measured by various vital measuring instruments, and the evaluation result of the level of consciousness obtained by observation of the subject's caregiver and the like with information of measurement date or acquisition date and time. The various information recorded in the information recording unit 4 can be input or corrected through the input unit 3a, the information transmitting and receiving unit 3c and the information input unit 24 (not shown) included in the tablet terminal 3. Moreover, content of the various information recorded in the information recording part 4 can be confirmed through the display unit 3b and the information transmitting and receiving unit 3c which are included in the tablet terminal 3.

The information recording unit 4 stores the personal information 7 of the subject, the measurement value of the vital signs measured by each vital meter, the evaluation result of the consciousness level obtained by observation for the subject, and vital information 8 including the information of the measurement date and time or acquisition date and time. In addition, the personal information 7 and the vital information 8 are configured to be recordable in association with identification information that can identify individual subjects. As a result, the plurality of subjects can be identified, and the plurality of subjects can use one health status determination device 1.

The vital information 8 includes measurement values of body temperature, pulse rate, systolic blood pressure, diastolic blood pressure, pulse pressure and respiratory rate, which are the classical vital signs. In addition, the vital information 8 includes a measurement value of oxygen saturation. Furthermore, the vital information includes the evaluation result of the aforementioned consciousness level. In addition, the classical vital signs may indicate that the oxygen saturation and the amount of urine are included in addition to the above contents.

In addition, the measurement date and time or acquisition date and time included in the vital information 8 are the date and time when the subject performed the vital measurement or the confirmation of the consciousness level. For example, the time when the subject performed the vital measurement by himself or the time when the caregiver observed the subject may be confirmed and then input.

In addition, the vital information 8 includes the value of the vital sign on which the score value information 103 was determined to be an abnormal value, based on the scoring reference information 102. That is, the vital information 8 includes not only the value of the vital sign when the score value information 103 is determined to be normal, but also the value of the vital sign when the score value information 103 is determined to be abnormal.

In addition, the vital information 8 includes a value in which the measurement value of the vital sign is determined to be an abnormal value, based on the vital determination reference information 102a. That is, the vital information 8 includes not only the value where the measurement value of the vital sign is determined to be normal, but also the value where the measurement value of the vital sign is determined to be abnormal.

Here, the vital information 8 does not necessarily include both the value of the vital sign which was the basis when the score value information 103 was determined to be abnormal and the value of the measured vital sign value that was determined to be abnormal. As will be described later, if the intra-individual variation unique to the individual is reflected as the vital information of the at least 30 pieces of measurement data, any one of the above-described vital sign values may be adopted. However, by including all of the abnormal values in the vital information 8, the variation of the method for identifying the variation in the individual increases, so that it can be appropriately selected. Therefore, it is preferable that the vital information 8 includes both the value of the vital sign which is the basis when the core value information 103 is determined to be abnormal and the value of the measurement value of the vital sign that is determined to be abnormal.

In addition, the type of vital information 8 is not necessarily limited to the measurement results of body temperature, pulse rate, systolic blood pressure, diastolic blood pressure, pulse pressure, respiratory rate, oxygen saturation, and evaluation result of consciousness level, and may include other vital signs and perform scoring to determine the score value. For example, the amount of urine, weight, pain (with/without pain, or degree), and other pathological abnormalities can be included in the vital information. However, the above-mentioned vital signs are the most representative vital signs, and the acquisition of the vital information is also easy and convenient, so it is preferable to be adopted. Furthermore, the measurement values of the classical vital signs, such as body temperature, pulse rate, systolic blood pressure, diastolic blood pressure, pulse pressure, and respiratory rate, which are obtained from the same subject, are based on the normal distribution, so that a scoring condition based on the normal distribution can be configured. Therefore, it is preferable to adopt especially the measurement values of the classical vital signs.

In addition, the vital meter for measuring the value of the vital sign in the vital information is not particularly limited, and it is sufficient if the body temperature, pulse, systolic blood pressure, diastolic blood pressure, respiratory rate and oxygen saturation can be measured. For example, the vitals may be measured using a vital meter for home. More specifically, it is not necessary to use a vital meter if the value of the vital sign can be measured. For example, it is also possible to measure the pulse rate or the respiratory rate for 1 minute while measuring the time with a clock, and use it as vital information. However, from the viewpoint of accurately grasping the fluctuations in the individual of the measurement values of body temperature, pulse rate, systolic blood pressure and diastolic blood pressure according to the normal distribution as the classical vital signs, the values of the vital signs are preferably obtained by the same technique. In the daily measurement, the type of vital meter is frequently changed, or the measurement by the vital meter and the measurement without the vital meter are mixed, so that differences arise depending on how the vital sign is measured. Therefore, it is desirable to measure the value of the vital sign using the same technique or the same vital meter as much as possible.

The vital information 8 is configured to be capable of being broadly recorded every second. In addition, the vital information 8 may be configured to be recorded at different time intervals, for example, every minute, every hour, etc.

Further, the vital information 8 may employ a configuration in which measurement values measured at irregular times are recorded instead of being measured at regular intervals. In addition, in the case of this irregular measurement, it is preferable to use a structure which records at least 30 pieces of vital information 8 in a fixed period, for example, at least 30 pieces of vital information 8 are acquired in one minute, at least 30 pieces of vital information 8 are acquired in 30 minutes, at least 30 pieces of vital information 8 are acquired in one hour, at least 30 pieces of vital information 8 are acquired in a few hours, at least 30 pieces of vital information 8 are acquired in a day, at least 30 pieces of vital information 8 are acquired in a few days, at least 30 pieces of vital information 8 are acquired in one week, at least 30 pieces of vital information 8 are acquired in several weeks, or at least 30 pieces of vital information 8 are acquired in one month.

Furthermore, the vital information 8 may be recorded as “the vital information 8 of 30 measurement data” by randomly extracting at least 30 pieces of data from the accumulated vital information regardless of a regular interval or irregular interval.

As described above, the vital information 8 is configured to be capable of recording at least 30 pieces of measurement data regardless of the length of time and the regularity of the measurement interval.

In addition, the information recording unit 4 is capable of recording the reference time information 9 which is information of the time at which the subject performs the measurement and acquisition of the vital information. In the reference time information 9, for example, a reference time for measuring and acquiring the vital information of the subject is recorded, such as 8:30 in the morning and 18:00 in the evening. The reference time information 9 can be freely configured and corrected.

In the information recording unit 4, posture information 10, which is information of a correct posture when measuring the value of each vital sign, is recorded. The posture information 10 is as follows, for example.

(1) Body Temperature

For example, when measuring body temperature with a thermometer that measures body temperature under the armpits, body temperature is information indicating a posture of “whether the thermometer is located at the center of armpits”, “whether armpits and thermometer are in close contact”, and “the same posture is taken each time” or the like.

(2) Pulse

For example, when measuring the pulse rate by applying an electronic pulse meter or fingers on the wrist, a pulse is information indicating a posture of “whether the subject is in a stable state”, “whether the subject has a relaxed posture”, and “whether the same posture is taken every time” or the like.

(3) Systolic Blood Pressure and Diastolic Blood Pressure

For example, when measuring in an oscillometric method, which measures the vibration of blood vessels, systolic blood pressure and diastolic blood pressure is information indicating the posture of, for example, “whether the subject is in stable state”, “whether a wound arm or wrist is at the height of the heart”, “whether the same posture is taken every time” or the like.

As will be described later, if a certain number of data (at least 30) is used in the calculation of the scoring condition by the reference calculation means and the scoring processing means, the vital mean value used for the calculation of the scoring condition, and the calculation of the vital reference deviation, is recorded, but the number of recording of vital information is not limited. In addition, the vital information 8 need not always be recorded at regular intervals, such as every second, every minute, every hour, or every day, and there may be times when the vital information 8 is not recorded. Here, from the viewpoint of properly grasping the intra-individual variation of the same individual, the aspect which records vital information every 1 second is generally good, and may be a structure in which the vital information is recorded once to 24 times a day.

In addition, the reference time information 9 does not necessarily need to be recorded in the information recording unit 4. However, by recording the reference time information 9, it is easy to measure and acquire vital information in a time zone deemed appropriate. In addition, when the reference time is reached, the fact may be notified of to the subject or the caregiver.

In addition, the posture information 10 does not necessarily need to be recorded in the information recording unit 4. However, by recording the posture information 10, the subject can be urged to measure at an appropriate posture while displaying the posture information 10 at the time of the measurement of each vital sign.

In addition, the content of the measurement method and the posture information 10 of each vital sign is not limited to the above descriptions, and the content of the vital measurement method and the suitable posture information 10 may be suitably changed.

The information recording unit 4 is capable of recording the temperature information 11 of a place where the vital information is measured and acquired. The temperature information 11 is recorded in association with the recording at the time of measurement or acquisition of the vital information 8. The temperature information 11 adopts the information which a subject confirms and inputs as the temperature of a measurement place, for example.

Here, it is not always necessary to make it possible to record the temperature information 11 of the place at which the vital information is measured and acquired in the information recording part 4. However, it is possible to confirm whether the environment in which the vital information was measured and acquired was an appropriate place, by recording the temperature information 11.

As shown in FIG. 4, the scoring reference information 102 serving as a reference when scoring each input vital information by the scoring processing means 100 is recorded in the information recording unit 4. In addition, score value information 103 which is information on the numerical value obtained as a result of scoring based on the scoring reference information 102 is recorded in the information recording unit 4.

In addition, score determination reference information 18 serving as a reference when the determination processing means 6 determines whether the score value information obtained from the content of the inputted vital information is an abnormal value is recorded in the information recording unit 4.

The scoring reference information 102 and the score determination reference information 18 to be described later can add or modify information via the input unit 3a of the tablet terminal 3, the information transmitting/receiving unit 3c, and the information input means 24 of the calculation unit 2. Further, the contents of each scoring reference information 102 may be confirmed via the display unit 3b of the tablet terminal 3. In addition, the detailed contents of each reference in the scoring reference configuring means 101 will be described later.

In the information recording unit 4, score determination result information 12, which is information of a determination result of determining whether the score value information 103 is an abnormal value by the determination processing means 6, is recorded. The content can be confirmed through the display part 3b of the tablet terminal 3. In addition, the score determination result information 12 may indicate the determination result by color coding according to the score as well as the display of abnormality or normality. For example, whether or not there is an abnormality can be indicated by color coding, such as red for 3 or more points, yellow for 2 points, no color for 1 or less points, or the like.

Further, the score determination result information 12 may be a result of determining not only the individual score value information but also the total score obtained by adding a plurality of (for example, all or a part) score value information 103. In this case, the determination result may be indicated by the determination of abnormality or normality or color classification according to the score for the total score obtained by adding a plurality of score value information 103.

In addition, in the information recording unit 4, vital determination reference information 102a serving as a reference when the determination processing means 6 determines whether the value of the input vital sign is an abnormal value is recorded.

The vital determination reference information 102a is capable of adding or modifying information via the input unit 3a and the information transmitting and receiving unit 3c of the tablet terminal 3, and the information input unit 24 of the calculating unit 2.

In the information recording unit 4, the vital determination result information 12a, which is information of the determination result when the determination processing means 6 determines whether the value is an abnormal value with respect to the value of the vital sign, is recorded. The content can be confirmed through the display part 3b of the tablet terminal 3.

In addition, in the information recording unit 4, the re-measured vital information 13, which is the vital information when the measurement and the like are re-performed in relation to the measurement and acquisition of the vital information and the date at the time of the measurement, can be recorded as the vital information 8. The re-measured vital information 13 may be, for example, vital information re-measured to confirm the accuracy of the vital information when the determination processing means 6 determines that the score value obtained for the vital information is an abnormal value.

In addition, when each vital information is displayed on the display unit 3b of the tablet terminal 3, characters representing vital information of three patterns can be displayed in different colors, with respect to normal vital information recorded without re-measurement, vital information targeted for re-measurement, and vital information after re-measurement.

Here, it is not necessary to make it possible to record the score determination result information 12 and the vital determination result information 12a in the information recording unit 4. However, it is preferable to enable recording of the score determination result information 12 and the vital determination result information 12a in the information recording section 4, in that it is possible to confirm the result of the determination of past vital information, to use it as reference information for improving the accuracy of the decision, to be used as a reference to the diagnosis result of the doctor or to be linked to the medical system.

Further, it is not necessary to allow the re-measured vital information 13 to be recorded in the information recording unit 4. However, since it is possible to verify whether the vital measurement is correct using the re-measured vital information 13, it is preferable that the re-measured vital information 13 can be recorded in the information recording unit 4.

[3. Reference Calculation Means]

A reference calculation means 5 will now be described. The reference calculation means 5 is one of the functions that the software to which the present disclosure is applied executes on the calculating part 2, and calculates the numerical range which becomes scoring reference information 102 for calculating score value information 103 with respect to the vital information recorded in the information recording unit 4 or calculates a vital mean value and a vital standard deviation used for calculation of the numerical range which becomes this scoring reference information 102. In the health status determining unit 1, the numerical range which becomes the scoring reference information 102 is calculated by the reference calculation means 5 about the measurement values of body temperature, pulse, systolic blood pressure, diastolic blood pressure, pulse pressure, and respiratory rate, and this becomes a reference when scoring.

Further, the reference calculation means 5 calculates the numerical range for vital determination which becomes vital determination reference information 102a for determining whether it is an abnormal value with respect to the vital sign value, or calculates a vital mean value and a vital standard deviation used for calculation of the numerical range for vital determination which becomes the vital determination reference information 102a, with respect to the vital information recorded (input vital information) in the information recording unit 4. In the health status determination device 1, the numerical value range for vital determination which becomes the vital determination reference information 102a is calculated by the reference calculation means 5 with respect to a measurement values of body temperature, pulse, systolic blood pressure, diastolic blood pressure, pulse pressure, and respiratory rate, and the numerical value range becomes a reference when determining whether the value of a vital sign is an abnormal value.

Various types of information calculated or recorded by causing the calculation unit 2 to function as the reference calculation unit 5 can be added or modified via the input unit 3a of the tablet terminal 3, the information transmission/reception unit 3c, and the information input unit 24 of the calculation unit 2. Further, the contents of the various information calculated or recorded by causing the calculation unit 2 to function as the reference calculation means 5 can be confirmed through the display unit 3b of the tablet terminal 3.

FIG. 4 shows a function by which the software to which the present disclosure is applied is executed in the calculation unit 2. The calculation unit 2 functions as a mean calculation means 14, a standard deviation calculation means 15, a normal distribution calculation means 16, a mode calculation means 110, a scoring reference configuration means 101, the vital reference setting unit 101, and a vital determination reference configuring means 101a which constitute the reference calculating unit 5.

The mean calculation means 14 and the standard deviation calculation means 15 calculate the “standard deviation of the vital information” in the distribution of the total vital information under the same condition as the “mean value of the vital information” under the same condition from the recorded information under a predetermined condition, respectively, based on the vital information 8 (measurement values of body temperature, pulse rate, systolic blood pressure, diastolic blood pressure and pulse pressure) recorded in the information recording unit 4 and its re-measured vital information 13. Further, in the following description, the mean value of the vital information is referred to as “the vital information mean value”, and the standard deviation of the vital information is referred to as “the vital information standard deviation”, except that the mean value or the name of the standard deviation of the type for performing a special calculation is indicated. In addition, predetermined conditions are mentioned later.

In addition, the mean calculation means 14 and the standard deviation calculation means 15 calculate, for the vital information 8 recorded in the information recording unit 4, the vital information mean value and the vital information standard deviation, including the value of the vital sign when the score value information 103 is determined to be an abnormal value, based on the score judgment reference information 18. In addition, the mean calculation means 14 and the standard deviation calculation means 15 calculate, for the vital information 8 recorded in the information recording unit 4, the vital information mean value and the vital information standard deviation, including the value of the vital sign when the value of the vital sign at the inputted determination time point is determined to be an abnormal value based on the vital decision reference information 102a.

In this way, by calculating not only the value of the vital sign that is determined to be normal but also the value of the vital sign that is determined to be abnormal, the mean value or the standard deviation reflecting the variation in the individual of the subject can be obtained. Further, by using such a mean value and a standard deviation, it is possible to create a reference that reflects the variation in the individual of the subject when configuring the scoring reference information 102 or the vital determination reference information 102a.

The mode calculation means 110 calculates the mode from the measurement value of the respiratory rate in the vital information under the predetermined conditions. The calculated mode is used to configure a numerical value that is the reference of the respiratory rate scoring condition. In addition, the calculated mode of the respiratory rate is recorded in the information recording unit 4 together with the calculation conditions (adopted conditions, period, date).

The “predetermined conditions” employed at the time of calculation of the mean calculation means 14, the standard deviation calculation means 15, and the mode calculation means 110 usually employ a method of using 30 pieces of vital information (measurement values of body temperature, pulse rate, systolic blood pressure, diastolic blood pressure, pulse pressure and respiratory rate) from the determination time point. The vital information of this period is the past 30 pieces of vital information 8 and re-measured vital information 13 including measurement data at the time of determination.

Here, the configuration of the past 30 pieces of vital information are the data of the vital information measured generally every second as mentioned above, and in addition, different lengths of time may be employed, such as data of vital information being measured every minute, several minutes, every hour, every day, or every month. In addition, it is also possible to extract the past 30 pieces of irregularly acquired data. At this time, a method may be used of extracting 30 pieces of vital information to go back the obtained turn. On the other hand, a method may be used of extracting 30 pieces of vital information by configuring certain extraction conditions for irregularly acquired data. As the extraction conditions, for example, a condition for extracting 30 pieces of vital information within a predetermined 1 hour range, or a condition in which an interval between acquisition times of vital information satisfies a certain condition (the interval is at least 5 minutes or 1 hour apart), may be considered. Further, a method may be used of selecting and extracting 30 pieces of vital information 8 randomly from the vital information 8 regularly measured at regular intervals. The extraction conditions for the past 30 pieces of vital information can be appropriately configured as necessary. In any extraction method, if at least 30 pieces of vital information 8 can be extracted, a normal distribution reflecting the variation in the individual can be obtained and used for the scoring reference information 102 or the vital decision reference information 102a.

As described above, the vital information 8 is configured to, broadly, be capable of being recorded every second. The vital information 8 may be configured to be recorded at different time intervals, for example, every minute, every hour. Furthermore, the vital information 8 may be configured to be measured and recorded irregularly several times a day. When the calculating unit 2 functions as the mean value calculation means 14, the standard deviation calculation means 15, and the mode calculation means 110 to calculate the vital mean value, the vital standard deviation, and the mode value, the vital mean value, the vital standard deviation, and the mode can be calculated appropriately, under the configuration conditions. For example, when the conditions for extracting 30 pieces of vital information are configured, the vital mean value, the vital standard deviation and the mode can be calculated from the extracted 30 pieces of vital information.

In addition, at the time of determination of the value of the score value information 103 or the value of the vital sign based on the input information of the person of the input, the mean value calculation means 14, the standard deviation calculation means 15, and the mode calculation means 110 refer to the vital information 8 and the re-measurement vital information 13 recorded before every determination time point, and calculate the mean value of the vital information, the standard deviation of the vital information, and the mode at the time of the determination. Thereby, the reference used by the determination processing means 6 (or the score processing means 100) is changed for each determination time point, and it is easy to reflect the intra-individual variation of the subject's vital information in the determination of whether the score value information 103 based on the vital information is an abnormal value and the determination of whether the value of the vital sign is an abnormal value.

In addition, the number of pieces of used vital information may be 30 pieces or more, and the configuration may use a larger number of pieces of the vital information 9, such as 90 or more. By increasing the number of pieces of vital information 8, it is possible to increase the accuracy of the contents of the normal distribution reflecting the variation in the individual. In addition, the number of pieces of data of 30 pieces or more is preferable as the minimum number for identifying the variation in the individual.

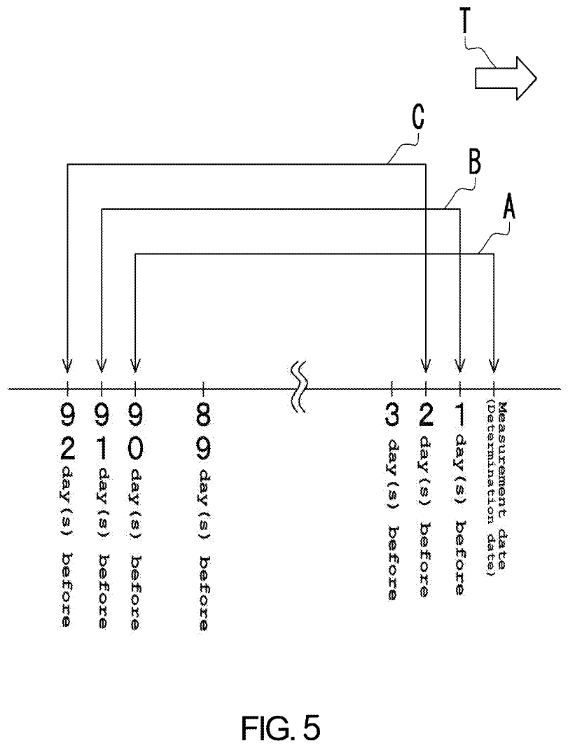

As the “predetermined condition” employed at the time of calculation of the mean calculation means 14, the standard deviation calculation means 15, and the mode calculation means 110, for example, when “for 90 days” is employed, this calculation period can be configured such that the range of 90 days moves by one day with the passage of time as shown in FIG. 5. That is, the period of 90 days used for calculation on a measurement day (decision date) is represented by the range (symbol A) from 90 days before a measurement day to the measurement day including the measurement day. In addition, the “predetermined condition” used for calculation 1 day before a measurement day is represented by the range (symbol B) from 91 days before a measurement day to the day before a measurement day. Further, the “predetermined condition” used for calculation two days before the measurement day is represented by a range (symbol C) from 92 days before the measurement day to two days before the measurement day. In this manner, the 90-day range of the “predetermined condition” can be set to move one day with the passage of time (the direction of the arrow of the symbol T). This point is the same even when used as the number of 30 pieces of data in different lengths of time (for example, minutes, hours, a day).

In addition, the “predetermined condition” employed at the time of calculation of the mean calculation means 14, the standard deviation calculation means 15, and the mode calculation means 110 is configured such that 30 pieces of vital information including the determination time point is used, but the determination time need not necessarily be a starting point. For example, a configuration in which 30 pieces of vital information is used starting from “data before determination time” except for the determination time point can also be adopted. However, it is preferable that 30 pieces of vital information is used for the “predetermined condition” adopted at the time of calculation of the mean value calculation means 14, the standard deviation calculation means 15, and the mode calculation means 110, in that it is possible to reflect the current state of the same individual and it is easy to grasp the variation in the individual by including the determination time point.

In addition, the “predetermined condition” employed at the time of calculation of the mean calculation means 14, the standard deviation calculation means 15, and the mode calculation means 110 does not necessarily need to be vital information measured on consecutive days (numbers). For example, in a case where there is a day when the subject does not perform the vital measurement (timing) and there is a day when the vital information is not recorded (timing), the number of days (number) of a predetermined condition may be “30 days (30 pieces).

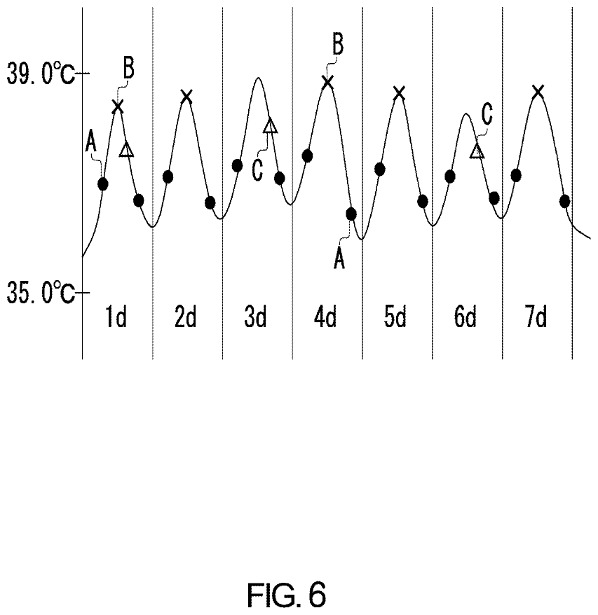

For example, as indicated by the symbol A (black circle) in FIG. 6, the vital information is recorded twice in the morning and afternoon every day, and all the information is used for the calculation of the mean value calculation means 14, the standard deviation calculation means 15, and the mode calculation means 110.

At this time, in the present disclosure, if the data number of the configured number of vital information is provided, it does not necessarily need to be the vital information continuously acquired every second, every minute, every hour, every day. Like the vital information shown by the symbol B (the figure of X) or the symbol C (white triangle) of FIG. 6, there may be an aspect in which the date (timing) of acquiring vital information is discontinuous and is acquired once in several days (several times). Furthermore, there may be an aspect in which information is partially extracted based on the set conditions in the state where continuous recording of vital information exists. The configured conditions are contents such as extracting only vital information on every Monday, only vital information acquired in the morning, extracting only a specified date, and the like.

The normal distribution calculation means 16 is a portion for calculating the normal distribution from the mean value and the standard deviation of the vital information under predetermined conditions. A normal distribution at each determination time point of the subject can be calculated, and a normal distribution curve obtained by graphing the established density function is created for the calculated normal distribution, and this normal distribution curve is displayed on the display section 3b of the tablet terminal 3. In addition, as described above, the mean value and the standard deviation of the vital information under the predetermined conditions are also calculated including the value of the vital sign which is the basis determined as abnormal. Accordingly, the normal distribution calculated by the normal distribution calculation means 16 is also created including the value of the vital sign used as the basis determined as abnormal.

The scoring reference configuring means 101 interworks with the mean value calculation means 14, the standard deviation calculation means 15, and the mode calculation means 110 to generate the scoring reference information 102 used for scoring by the scoring processing means 100, based on the vital mean value, the vital standard deviation and the mode value calculated from each calculating part. The generated scoring reference information 102 is recorded in the information recording unit 4.

More specifically, the scoring reference configuring means 101 interworks with the mean value calculation means 14, the standard deviation calculation means 15, and the distribution calculation means 16 to generate the scoring reference information 102 used for scoring, in reference to the measurement values of body temperature, pulse, systolic blood pressure, diastolic blood pressure, and pulse pressure measured from the subject, based on the vital mean value and the vital standard deviation calculated from the respective calculation means.

In addition, in the creation of the scoring reference information 102 as described above, the vital information 8 contains the value of the vital sign when the score value information 103 is determined to be an abnormal value (or the value of the vital sign when the value of the vital sign at the time of input determination is determined to be an abnormal value).

In addition, the scoring reference configuring means 101 interworks with the mode calculation means 110 to generate the scoring reference information 102 used for scoring by the scoring processing means 100, in reference to the measurement value of respiratory rate measured from the subject, based on mode calculated from the mode calculation means 110. In addition, it may be an aspect in which the mean is used instead of the mode.

In addition, the scoring reference information 102 includes not only the calculation result of each calculation means but also information of a predetermined fixed numerical range used when scoring the measurement value of oxygen saturation, or a predetermined observation state capable of distinguishing the degree of consciousness level.

More specifically, for the measurement value of oxygen saturation measured from the subject, the predetermined numerical range can be input from the input part 3a of the tablet terminal 3, and can be configured as the scoring reference information 102. The configured scoring reference information 102 is recorded in the information recording unit 4.

In addition, for the evaluation result of the consciousness level acquired from the subject, the content of the predetermined observation state which can distinguish the grade of consciousness level may be input, and can be configured as the scoring reference information 102. The set scoring reference information 102 is recorded in the information recording section 4. In addition, the details of the calculation of the vital mean value, the vital standard deviation, the mode and the scoring reference information 102, and the setting of the scoring reference information 102 composed of a plurality of items will be described later.

The vital determination reference configuring means 101a interworks with the mean value calculation means 14, and the standard deviation calculation means 15 to generate the vital decision reference information 102a used in decision of the vital sign by the decision processing means 6, based on the vital mean value and the vital standard deviation calculated from each calculation means. The generated vital determination reference information 102a is recorded in the information recording unit 4.

More specifically, the vital determination reference configuring means 101a interworks with the mean value calculation means 14, the standard deviation calculation means 15, and the normal distribution calculation means 16 to generate the vital decision reference information 102a used in decision of the vital sign, for the measurement values of body temperature, pulse, systolic blood pressure, diastolic blood pressure, pulse pressure measured from the subject, based on the vital mean value and the vital standard deviation calculated from each calculation means.

Here, as described above, in the creation of the vital judgment reference information 102a, the vital information 8 includes the value of the vital sign when the value of the vital sign at the input determination time point is determined to be an abnormal value (or the value of the vital sign when the score value information 103 is determined to be an abnormal value).

[4. Scoring Processing Means]

The scoring processing means 100 will now be described. The scoring processing means 100 is one of the functions that the software to which the present invention is applied, executed in the operation unit 2, and calculates score value information 103 (information of score) according to the contents of the vital information, for the vital information at the determination time point input through the input part 3a of the tablet terminal 3, based on the processing information of the mean value calculation means 14, the standard deviation calculation means 15 and the mode calculation means 110, or the scoring reference information 102 including the preset criteria.

The score value information 103 calculated by the scoring processing means 100 is recorded in the information recording unit 4, as described above. At this time, the score value information 103 is recorded in association with the identification information which can identify an individual and the information used as the calculation standard of a score value. The scoring processing means 100 is configured to calculate the score value information 103 in conjunction with the information recording unit 4 and the reference calculation means 5.

In addition, the content of the score value information 103 can be confirmed through the display unit 3b of the tablet terminal 3. In addition, the score value information 103 may be confirmed on these screens and the like by sending score determination result information 12 to an external server or an external terminal through the information transmitting and receiving unit 3c of the tablet terminal 3 as well as the display unit 3b of the tablet terminal 3.

[5. Determination Processing Means]

The determination processing means 6 will now be described. The determination processing means 6 is one of the functions that the software to which the present invention is applied executes in the calculation unit 2, and determines whether the score value information 103 is an abnormal value with respect to the score value information 103 in which the vital information at the determination time point input through the input unit 3a of the tablet terminal 3 is scored by the scoring processing means 100, based on the score determination reference information 18. In addition, the determination processing means 6 determines whether the value of the vital sign is an abnormal value, for the value of the vital sign at the input judgment time point, based on the vital determination reference information 102a.

The score determination result information 12 and the vital determination result information 12a, which are the determination results determined by the determination processing means 6, are recorded in the information recording unit 4. In addition, the contents of the score determination result information 12 and the vital determination result information 12a may be confirmed through the display unit 3b of the tablet terminal 3. In addition, the contents of the score determination result information 12 and the vital determination result information 12a may be transmitted to an external server or an external terminal through the information transmission/reception unit 3c of the tablet terminal 3 as well as the display unit 3b of the tablet terminal 3, and confirmed on these screens and the like.

In addition, the contents of the score determination result information 12 and the vital determination result information 12a may be not only displayed on the display portion 3b of the tablet terminal 3, but may also be notified of by a notification sound or a mail message which notifies that the score determination result information 12 and the vital determination result information 12a have been processed. When notifying of the score determination result information 12 and the vital determination result information 12a by the notification sound, it is also possible, for example, to change the type of the notification sound when the content is abnormal.

Next, a description will be given of specific contents of a device and an input screen to be used when executing the software to which the present invention is applied.

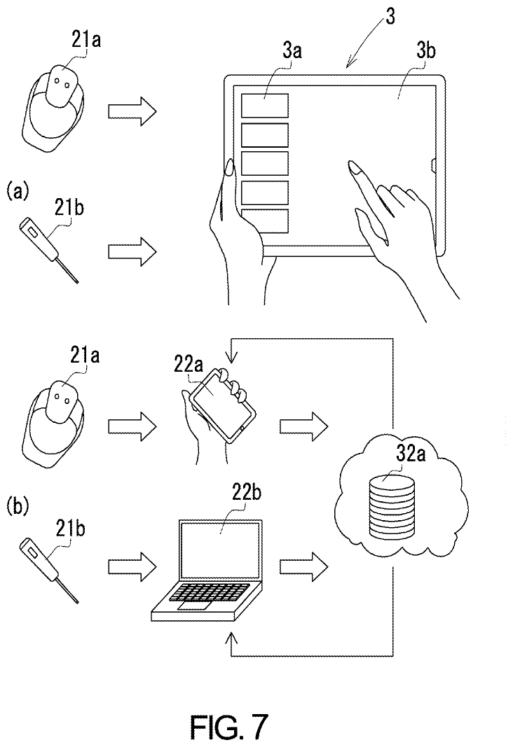

For example, as shown in FIG. 7B, the vital information is acquired by a wearable vital meter 21a, a thermometer 21b, or the like, and the measurement values measured thereby are input via the screen displayed on the display part 3b of the tablet terminal 3 with the information of the measured time. On the display unit 3b, an input unit 3a in the form of a touch panel is displayed, and vital information is input thereto. If the tablet terminal 3 to which the software to which the present invention is applied is introduced (first system configuration), the terminal itself can display the recording of information, the determination of the state of health and the determination result.

In addition, as shown in FIG. 7B, it is also possible to access the information management server 32a, which is the external server described in the above-described second system configuration, from a smart phone terminal 22a or a personal computer terminal 22b (hereinafter, referred to as a “PC terminal 22b”) to input vital information through the smart phone terminal 22a or the PC terminal 22b. Based on the vital information transmitted from each terminal, the determination of the state of health is made in the information management server 32a, and the result information is transmitted to each terminal, and the result information is displayed on the screen of each terminal.

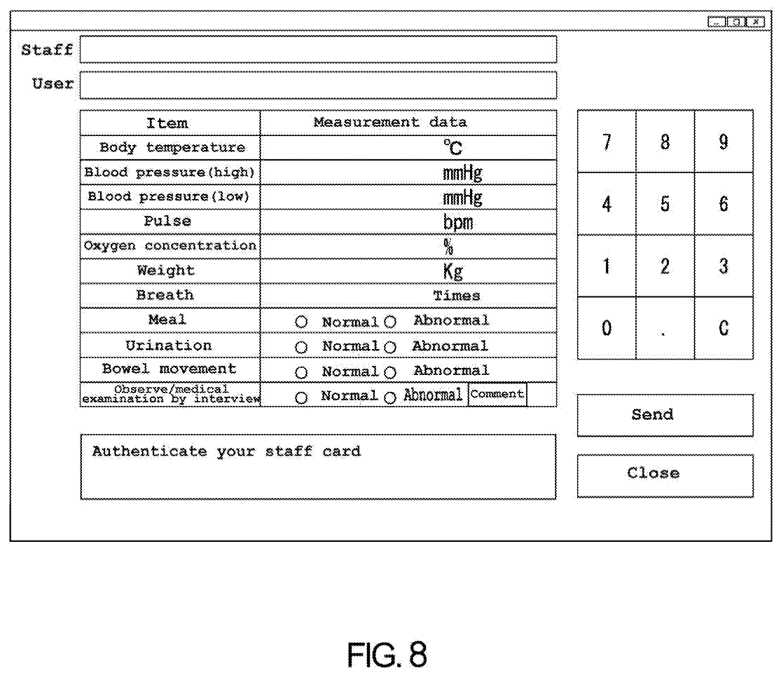

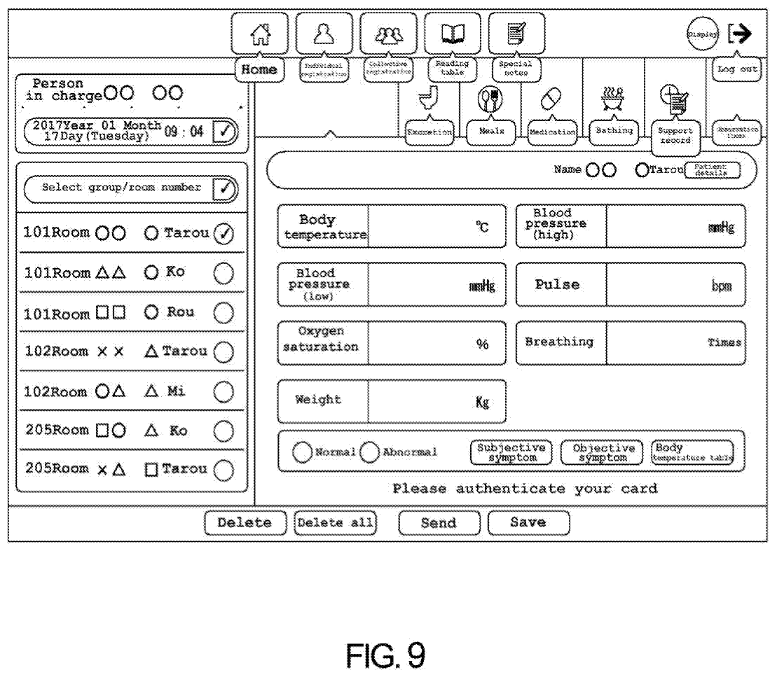

Moreover, the screen shown in FIG. 8 and FIG. 9 is shown as an input screen of the tablet terminal 3, the smart phone terminal 22a, and the PC terminal 22b. FIGS. 8 and 9 are examples of input screens used when a patient in a hospital or a resident, such as a nursing facility, is to be determined as a health object. In FIG. 8, the input item of one person and the ten-key area which displays numbers are displayed. The name display column of the subject and the staff in charge, and input fields of measurement data in body temperature, blood pressure (up and down), pulse rate, oxygen concentration, weight, and respiratory rate are provided. As for the value of each vital sign, input to the ten-key area can be achieved by a touch panel or a cursor operation on the screen.

In addition, in the screen display of FIG. 8, items of diet, urination, bowel movement, and observation/check-up are provided, and in addition to the value of the vital sign, a plurality of items for confirming the health status of the subject are also provided. By recording a plurality of these items identifying health conditions, it is possible to make a record of the subject's daily health conditions. The input information is recorded in the information recording unit 4 inside the device by touching or clicking the transmit button and transmitting the information to an external information management server 32a (information recording unit of the server).

In the input screen illustrated in FIG. 9, input fields of measurement data of a plurality of vital signs and selection items of normal or abnormal conditions determined by the subject on their own are provided on the right side of the screen. In addition, subjective sign, objective sign, and a thermal table can be selected to input additional conditions of information or to identify changes in the vitals of the subject.

In addition, in the screen of FIG. 9, the names of the plurality of subjects are displayed, and the screen of the selected subject can be displayed by selecting the name field. Moreover, the information of the time at the time of input of the value of a vital sign is input simultaneously. Furthermore, in addition to the input screen of the value of the vital sign, it is also possible to record or display information on items of information registration, and information on care items such as excretion and meals to be provided.

As described above, the input screen when using the software of the present invention can be used for the patient of a hospital, a care facility, or the like, so that input or information can be displayed together with related items. In addition, the display of the input screen is not limited to the contents associated with the caregiver and the like, and is, for example, a screen configuration that combines input of the value of each vital sign and management of information such as record and weight as application software for health care. That is, it can also be set as the aspect in which a healthy subject uses for daily health care.

Next, the method of specific determination about the score value information obtained from the vital information is demonstrated.

[6. About Calculation of Vital Mean Value, Scoring and Abnormality Determination]

[6-1. About Measurement of Body Temperature, Pulse, Systolic Blood Pressure, Diastolic Blood Pressure, Pulse Pressure]

The vital mean value and the vital standard deviation are calculated by the calculation unit 2 functioning as the mean value calculation means 14 and the standard deviation calculation means of the reference calculation means 5, based on the vital information 8 and the re-measurement vital information 13 recorded in the information recording section 4. In addition, the scoring reference information 102 and the vital determination reference information 102a for the measurement values of the body temperature, pulse, systolic blood pressure, diastolic blood pressure, and pulse pressure are configured based on the vital mean value and the vital standard deviation.

As a method for configuring the vital mean value, the vital standard deviation, the scoring reference information 102, and the vital determination reference information 102a based on the vital mean value and the vital standard deviation, there may be a method of using the vital information 8 and the re-measurement vital information 13 recorded in the information recording section 4 to calculate the vital mean value, and the like. In this method, the standard deviation based on the vital mean value and the distribution of the vital information is calculated using the following equations (3) and (4) in the mean value calculation means 14 and the standard deviation calculation means 15:

μ=(1/N)×ΣSi Equation (3); and

σ=√((1/N)×Σ(Si−μ)2) Equation (4).

Here, μ is the mean value of the vital information, Si is the measurement value of each vital information, N is the number of data of the entire vital information, and σ is the standard deviation. ΣSi represents the sum of measurement values of all vital information. In addition, the measurement value of each vital information is the value of the vital information acquired on the predetermined condition set as mentioned above. In addition, the content of all vital information here may be that extracted as a part of the information recorded in the information recording part 4 as mentioned above. The vital information herein is measurement values of body temperature, pulse rate, systolic blood pressure, diastolic blood pressure, and pulse pressure.

At any judgment point, when determining the vital information of the subject, the vital mean value μ and the vital standard deviation σ are calculated from the data of the same subject recorded in the information recording unit 4 using the above equations 3 and 4. That is, the scoring reference information 102 and the vital determination reference information 102a are calculated including the value of the vital sign which is the target of the determination measured at the determination time point. Subsequently, the scoring reference setting means 101 and the vital judgment reference setting means 101a use the values represented by the following equation (1) or (2) as the scoring reference information 102 and the vital judgment reference information 102a:

μ−nσ Equation 1; and

μ+mσ Equation 2.

Here, n and m are numbers greater than zero.

In the scoring reference information 102, the value shown by said Equation (1) and Equation (2) and the predetermined score value, i.e., the information of the score of 0-3 points are combined. This combination is as shown in Table 2 below.

TABLE 2

| Score | 3 | 2 | 1 | 0 | 1 | 2 | 3 |
|---|---|---|---|---|---|---|---|
| Systolic Blood | −3σ< | −3σ~−2.5σ | −2.5σ~−2σ | Within ±2σ | +2σ~+2.5σ | +2.5σ~+3σ | +3σ> |
| Pressure (mmHg) | | | | | | | |
| Diastolic blood | −3σ< | −3σ~−2.5σ | −2.5σ~−2σ | Within ±2σ | +2σ~+2.5σ | +2.5σ~+3σ | +3σ> |
| pressure (mmHg) | | | | | | | |
| Pulse pressure | −3σ< | −3σ~−2.5σ | −2.5σ~−2σ | Within ±2σ | +2σ~+2.5σ | +2.5σ~+3σ | +3σ> |
| (mmHg) | | | | | | | |
| Pulse rate | −3σ< | −3σ~−2.5σ | −2.5σ~−2σ | Within ±2σ | +2σ~+2.5σ | +2.5σ~+3σ | +3σ> |
| (times/minute) | | | | | | | |
| Body tempera- | −3σ< | −3σ~−2.5σ | −2.5σ~−2σ | Within ±2σ | +2σ~+2.5σ | +2.5o~+3σ | +3σ> |
| ture (° C.) | | | | | | | |
| Oxygen | 84 or | 85~89 | 90~92 | 93~100 | — | — | — |
| Saturation(%) | less | | | | | | |
| Respiratory | Mode −10< | Mode −6~−9 | Mode −5 | Within | Mode +5 | Mode +6~9 | Mode +10> |
| rate (respira- | | | | Mode ±4 | | | |
| tory rate/minute) | | | | | | | |
| Consciousness | Uncon- | Response | Abnormal | Normal | — | — | — |
| Level | sciousness | to pain | | | | | |

In addition, in Table 2, “−3σ” is a value of “μ−3σ” based on equation 1, “−2.5σ” is a value of “μ−2.5σ” based on equation 1, “−2σ” is a value of “μ−2σ” based on equation 1, “+3σ” is a value of “μ+3σ” based on equation 2, “+2.5σ” is a value of “μ+2.5σ” based on equation 2, and “+2σ” is a value of “μ+2σ” based on equation 2. In addition, μ and σ are the values computed from the measurement value of each vital sign measured on predetermined conditions (for example, 30 pieces of vital information).

As shown in Table 2, when scoring the measurement values of body temperature, pulse rate, systolic blood pressure, diastolic blood pressure, and pulse pressure with each score value of 0 to 3 points based on the contents thereof, the “μ±2σ, μ±2.5σ and μ±3σ” are calculated based on the equations 1 and 2.

More specifically, if the measurement value of the input vital sign is, in the vital mean value and the vital standard deviation calculated at the determination time, a value belonging to the range of “within μ±2σ”, it will be a score of 0 point, a value belonging to a range of “μ−2.5σ (or greater)˜(under) μ−2.5σ” or a range of “μ+2σ (or greater)˜(under) μ+2σ”, it will be a score of 1 point, a value belonging to a range of “μ−3σ(or greater)˜(under) μ−2.5σ” or a range of “μ+2.5σ(exceed)˜(within)μ+3σ”, it will be a score of 2 points, a value belonging to a range of “(under) μ−3σ” or “μ+3σ(exceeds)”, it will be a score of 3 points.

As for scoring of the measurement value of the input vital sign, the reference for each determination time point is configured by the vital mean value and the vital standard deviation calculated at the determination time point. In addition, the measurement values of body temperature, pulse rate, systolic blood pressure, diastolic blood pressure, and pulse pressure are classical vital signs according to a normal distribution, and the scoring reference information 102 calculated based on Equations (1) or (2) is a reference in which the subject's individual variation is reflected, and is a reference set based on the subject's normal distribution. Therefore, it becomes an index which can grasp the variation of the condition of a subject correctly.

Moreover, the determination processing means 6 designates “caution” when one point is calculated with respect to the score value information 103, and designates “warning” when two or more points are calculated. When the score value information 103 is 0, the judgment result of “caution” or “warning” does not appear, and it can be seen as a “normal” state. In other words, when a determination is made that the value of one or more vital signs is a value of one or more points, the judgment can be made as an abnormality divided into two stages of “caution” and “warning”. This content is score determination criteria information 18.

Further, the determination processing means 6 determines that the value of “μ±2σ or more” is equal to or more than (the value of the vital sign) with respect to the value of the vital sign (measurement value of each vital sign).

Further, the score value information 103 calculated from the values of the vital signs, the score determination result information 12 such as attention to this value, and the vital determination result information 12a are recorded in the information recording section 4 in association with the subject.

In addition, when the determination processing means 6 has made the determination of “warning” for the score value information 103 or the determination of “warning” for the value of the vital sign, the health status management device 1 may transmit an alarm sound or transmit an e-mail indicating that a “warning” has been made through the information transmitting/receiving unit 3c. Thereby, the caregiver etc. can be notified that an abnormality has occurred in the condition of the subject. In addition, here, the determination of the score value information 103 may be mainly configured to generate a warning sound or transmit an e-mail to an external terminal or the like when the report on the determination of the “warning” for the score value information is performed and the “warning determination” is performed.

Here, although n in the above Equation (1) or (2) has been described as being greater than 0, the numerical values of n and m are not limited to “2, 2.5 and 3” as described above, and the numerical value can be changed suitably and can be used as the scoring reference information 102.

In addition, the measurement values of body temperature, pulse rate, systolic blood pressure, diastolic blood pressure, and pulse pressure do not necessarily need to have the same as n and m in the Equation (1) or the Equation (2). Depending on the type of vital sign, the numerical values to be set to n and m may be different.

In addition, in the scoring reference information 102 shown in Table 2, as a range which distinguishes the score value information of one point and the score value information of two points, for example, the range of “within μ±2σ” and “μ+2σ (or greater)˜(less than) μ+2.5σ” is configured. That is, before and after the numerical value of μ+2σ, within 0 μ+2 σ and 0 μ+2 σ, it becomes one point. However, the configuring of the range is not necessarily limited to this content. For example, it can also be set as the content which becomes 0 points less than μ+2 σ, and one point more than μ+2 σ. In addition, the same applies to other numerical values.

In addition, in the scoring reference information 102 shown in Table 2, although the score value information 103 is configured in the range of 0 to 3 points, it is not necessarily limited to this range. For example, it is also possible to change the score value information into a setting that scores in a range of 0, 1 and 2 points. Furthermore, it is also possible to employ a value larger than three points. It goes without saying that the scoring criteria information 102 can be set appropriately so as to change the score value information 103. This also applies to scoring of oxygen saturation, respiratory rate, and consciousness level described later.

In addition, the numerical value which the determination processing means 6 judges abnormally about the score value information 103 is not limited to one or more points. For example, a determination using two points or more may be adopted. In addition, it is not necessary to judge the above determination in two steps of “caution” and “warning.” For example, there may be a configuration for dividing the determination into three or more stages, or an aspect for designation simply as one stage of “abnormal”. However, it is possible to distinguish the degree of abnormality of the score value information 103 by determining the above judgment in two stages of “caution” and “warning”, so that the countermeasures according to the degree of “caution” or “warning” are easily set. It is preferable to divide the above determination into two stages. This also applies to scoring of oxygen saturation, respiratory rate, and consciousness level described later.

In addition, although the determination processing means 6 is set to perform determination of whether it is an abnormal value with respect to the score value information 103 based on the measurement value of each vital sign, it does not necessarily need to be set in this way. For example, it may be set as an aspect which determines whether it is an abnormal value with respect to the “total points” of the score value information 103 based on several types of vital signs. In addition, it is also possible to provide an aspect in which specific types of vital signs (e.g., body temperature and pulse rate) are combined to determine whether the value is abnormal for the “total points” of the score value information 103 based on the combined vital signs. However, by determining whether or not the score value information 103 based on the measurement values of each vital sign is an abnormal value, it is easy to know the vital sign that was based on the above and this may become easy reference information in the response after the judgment. It is preferable that the score value information 103 based on the measured value of one vital sign is determined to be abnormal value. This also applies to scoring of oxygen saturation, respiratory rate, and consciousness level described later.

In addition, for example, “caution” or “warning” is configured for the “total point” of the score value information 103 based on a plurality of types of vital signs according to the score, and this “caution” or “warning” is set. It may be set as the aspect in which displays on the display part 3b, or rings an alert.

Incidentally, in the scoring criteria information 102 shown in Table 2, systolic blood pressure, diastolic blood pressure, pulse pressure, pulse rate, body temperature, oxygen saturation level, respiratory rate, and consciousness level can be cited as targets to be scored (markers), This is merely one example. In addition, the threshold value which distinguishes the score in scoring reference information 102 is also an example.

That is, the threshold value for distinguishing the type or score of the marker can be configured differently according to the type of the disease or the nature of the subject. For example, in the case of a subject having heart failure and a subject having a urinary tract infection, the threshold value for distinguishing the type or score of the marker may be configured differently.

Further, as the marker, there may be a case where only systolic blood pressure is employed in the blood pressure or both systolic blood pressure and diastolic blood pressure are employed. In addition, for example, in the case of a subject who is a normal person and a subject who is an elderly person having a disease, the threshold value for distinguishing the type or score of a marker may also be configured differently.

In addition, the scoring criteria information 102 also includes an aspect of scoring, including markers such as a history of the subject, a family history of the subject's family and relatives, and lifestyle.

In this case, for example, when performing scoring to determine the degree of heart failure for a subject with a history of heart disease or a subject having a history of heart disease in their family, a score is given to a marker of history or family history and score value information 103 is added to the total point. In addition, for example, for a subject who has a lifestyle of smoking, a score is given to the lifestyle marker and is added to the total points of the score value information 103.

Here, the difference between the case where the distribution of vital information based on the information of another individual is created using the vital information of a plurality of subjects and the case where the distribution of vital information of the same individual is created using the vital information of the same subject will be explained.

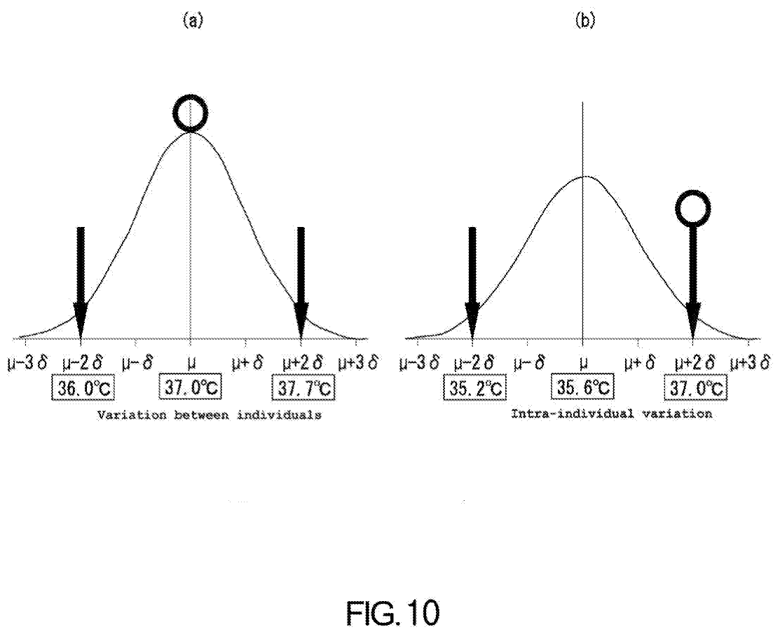

FIGS. 10A and 10B are graphs of a normal distribution curve created based on body temperature information. In FIGS. 10A and 10B, the horizontal axis represents a probability variable of body temperature, and the vertical axis represents a probability density. FIG. 10A is made up of many subjects, and FIG. 10B is made up of only the same subjects. In FIG. 10A, persons with various fluctuations and body temperature fluctuations are included, and the mean value μ is 37.0° C., which is the mean value of a plurality of subjects, the value of μ+2σ is 37.7° C., and the value of μ−2σ is 36.0° C.

However, in FIG. 10B, the vital information of the same individual is recorded, and since the fluctuation and fluctuation of body temperature peculiar to the person, the mean value μ is 35.6° C., the value of μ+2σ is 37.0° C., and the value of μ−2σ is 35.2° C.

That is, if the reference value stabilized to a certain score value at the time of scoring using each distribution is configured to μ+2σ, in the case of FIG. 10A, the body temperature of 37.0° C. corresponds to the position of μ (black circle in FIG. 7A). On the other hand, in FIG. 10B, the body temperature at 37.0° C. becomes the position of the upper limit of μ+2σ (black circle in FIG. 10B). That is, in the distribution shown in FIG. 10A and the distribution shown in FIG. 10B, the values of the same μ+2σ on the distribution become completely different value. Therefore, the scoring reference information 102 and the score value information 103 are also changed, and the determination result is also changed. In other words, in performing the determination of the subject of FIG. 10B, the scoring reference information 102 or the score value information 103, based on the vital information of the plurality of subjects cannot be used to grasp and “abnormal value”. Using the vital information of a large number of persons as a reference is not only a determination in the “inter-individual variation” which has been conventionally performed, but it shows that the “intra-individual variation” is effective in order to see the variation of the vital information peculiar to the subject.

In addition, the subject who exhibits the mean value or fluctuation of the body temperature shown in FIG. 10B does not correspond to a special case. In addition, a phenomenon unique to the subject may also occur in other vital signs such as systolic blood pressure, diastolic blood pressure, pulse rate, and respiratory rate, which are not caused by body temperature, and these are subject to normal distribution. As an example of the above-mentioned body temperature, there are many elderly people whose body temperature changes in the temperature range shown in FIG. 10B, and “intra-individual fluctuation” is effective when performing the vital signs for the determination of the health status of the elderly.

[6-2. About Measurement Value of Oxygen Saturation]

As a configuration method of the scoring reference information 102 about the measurement value of the oxygen saturation measured from the subject, the information of a fixed numerical range is configured as a reference. In the content shown in Table 2, when scoring with the score value of 0-3 points with respect to the measurement values of oxygen saturation degree, “93-100(%)” is configured as a score of 0 point, “90-92(%)” is configured as a score of one point, “85-89(%)” is configured as a score of two points, and “84(%) or less” is configured as a score of three points.

The score value information 103 of 0-3 points is calculated based on the scoring reference information 102 shown in Table 2 about the measurement value of oxygen saturation input. Incidentally, the determination as to whether it is an abnormal value by the determination processing means 6 for the score value information 103 is as described above.

In addition, score value information 103 calculated from the measurement value of oxygen saturation and score determination result information 12 such as attention to this value are recorded in the information recording unit 4 in association with the subject.

Here, the content of the scoring reference information 102 for the oxygen saturation shown in Table 2 is not limited thereto. The numerical range which divides the score value information of 0-3 points can be set as the scoring reference information 102 by suitably changing a configuration.

[6-3. About Measurement Value of Respiratory Rate]

As a method of setting the scoring reference information 102 for the measurement value of the respiratory rate measured from the subject, a method of using the vital information 8 and the re-measurement vital information 13 recorded in the information recording unit 4 for the calculation of the mode can be mentioned. In this method, the mode calculation means 110 calculates the mode with respect to the measurement value of the respiratory rate under predetermined conditions (e.g., 30 minutes). In addition, the measurement value of respiratory rate can adopt the value of the respiratory rate measured on the conditions set. In addition, the content of all vital information here may extract a part of the information recorded in the information recording part 4 as mentioned above.

At any determination time point, when determining the respiratory rate of the subject, starting from the judgment point, the mode is calculated from the data of the same subject recorded in the information recording unit 4 from the determination time point. That is, the scoring reference information 102 is calculated including the value of the respiratory rate which is the object of determination measured at the determination time point. The scoring reference setting means 101 configures the scoring reference information 102 from the mode so as to be the contents shown in Table 2.

In the content shown in Table 2, the reference configured based on the mode of respiration rate in predetermined conditions is shown. When scoring with each score value of 0 to 3 points for the respiratory rate, the “mode±4 (respiration rate/minute)” is set as the score of 0 point, “mode−5 (respiration rate/minute)” or “mode+5” (respiratory rate/minute) is set as a score of 1 point, “mode−6˜mode−9 (respiration rate/minute)” or “mode+6˜mode+9 (respiration rate/minute)” is set as a score of 2 points, “mode−10 (respiration rate/minute) or less” or “mode+10 (respiration rate/minute) or more” is set as score of 3 points.

The mode is calculated with respect to the measurement value of the input respiratory rate, and based on this mode, it becomes the scoring reference information 102 shown in Table 2, and the score value information 103 of 0-3 points is computed. Incidentally, the determination as to whether or not it is an abnormal value by the determination processing means 6 for the score value information 103 is as described above.

In addition, score value information 103 calculated from the measurement value of the respiratory rate and score determination result information 12 such as attention to this value are recorded in the information recording section 4 in association with the subject.

Here, the content of the scoring reference information 102 for the respiratory rate shown in Table 2 is not limited to this. The numerical range which divides the score value information of 0-3 points can be set as the scoring reference information 102 by suitably changing a configuration.

In addition, the scoring reference information 102 for the measurement value of the respiratory rate may use a value of the mean μ of the predetermined condition instead of the mode of the predetermined condition. The value of the mean μ can be calculated by the mean value calculation means 14. In this case, a score can be adopted as a reference by combining predetermined numerical value with the mean μ when setting the scoring reference information 102.

[6-4. About Consciousness Level]

A caregiver or the like may check the level of consciousness of the subject and apply the acquired result to predetermined observation information set as the scoring reference information 102. Confirmation of consciousness level may use existing AVPU assessments.

In the AVPU evaluation, Normal (Awake alert, A: alert), Abnormal (Respond by words but no subject, V: verbal), Respond to pain (Respond only to pain, P: Pain), and Unconsciousness (not responding to words or pain, U: Unresponsive) are set as predetermined observation states. A caregiver or the like observes the subject to determine which item of the AVPU evaluation corresponds to the item, and inputs the result through the input unit 3a or the like.

Scoring criteria information 102 for the level of consciousness is set to the content shown in Table 2. In Table 2, a normal score is 0, abnormality is a score of 1, painlessness is a score of 2 points, and unconsciousness is set to a score of 3 points. The scoring processing means 100 calculates the score value information 103 based on the information input by the caregiver or the like. Incidentally, the determination as to whether or not it is an abnormal value by the determination processing means 6 for the score value information 103 is as described above.

In addition, score value information 103 calculated from the evaluation result of the subject's consciousness level and score determination result information 12 such as attention to this value are recorded in the information recording unit 4 in association with the subject.

Here, the content of the scoring criteria information 102 regarding the evaluation result of the consciousness level of the subject shown in Table 2 is not limited to this. Consciousness assessment techniques other than AVPU assessment may be employed. In addition, the observation state which divides the score value information of 0-3 points may be suitably changed, and can be configured as the scoring reference information 102.

In the above description, scoring is performed using the body temperature, pulse rate, systolic blood pressure, diastolic blood pressure, pulse pressure, oxygen saturation, respiratory rate and the result of consciousness level among vital signs, and whether the calculated score value information 103 is abnormal is determined. Here, the vital sign of the subject need not necessarily be limited to these contents. For example, as a subject to be scored, it is also conceivable to employ urine volume, weight, pain (with or without pain), and other pathological abnormalities obtained from the subject as vital sign information.

[7. Creation of Display Information]

In the health status determination device 1 to which the present disclosure is applied, the content of the subject's vital information can be displayed as a normal distribution curve. It is also possible to display the vital information of the subject as a column table.

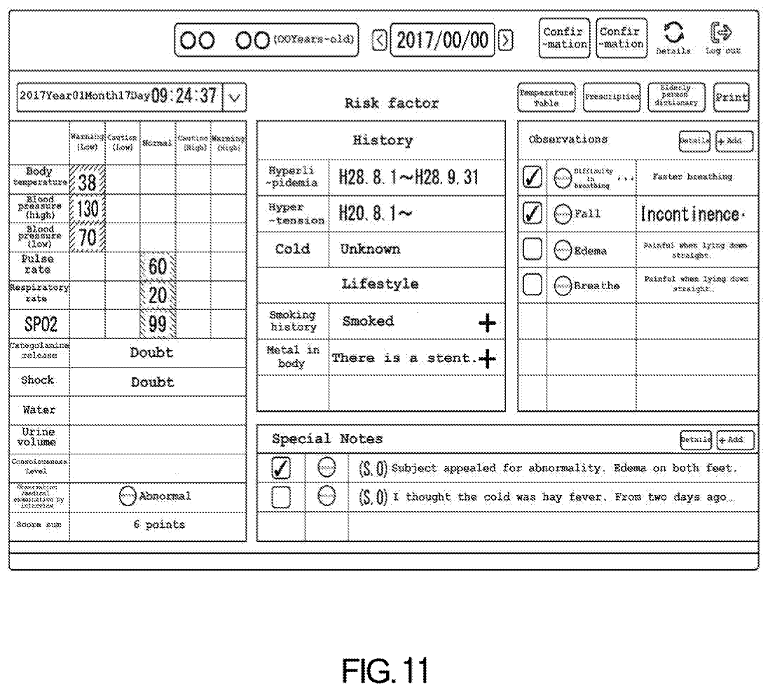

FIG. 11 shows an example of the column table. FIG. 11 shows vital information at the time of determination concerning a subject, information on whether the value of the score value information based on the content of the vital information is an abnormal value (warning, caution, normal information), information on the presence or absence of abnormality due to the observation or questionnaire result of the subject, and information of total points of sore value information.

In addition, in the thermal table shown in FIG. 11, the information on the past history, which is a risk factor of the health condition of the subject, and the information on lifestyle are displayed. In addition, the thermal table displays detailed observation information of the subject and information on special items. The information displayed on the thermal table can be created based on the information input via the input unit 3a or the like.

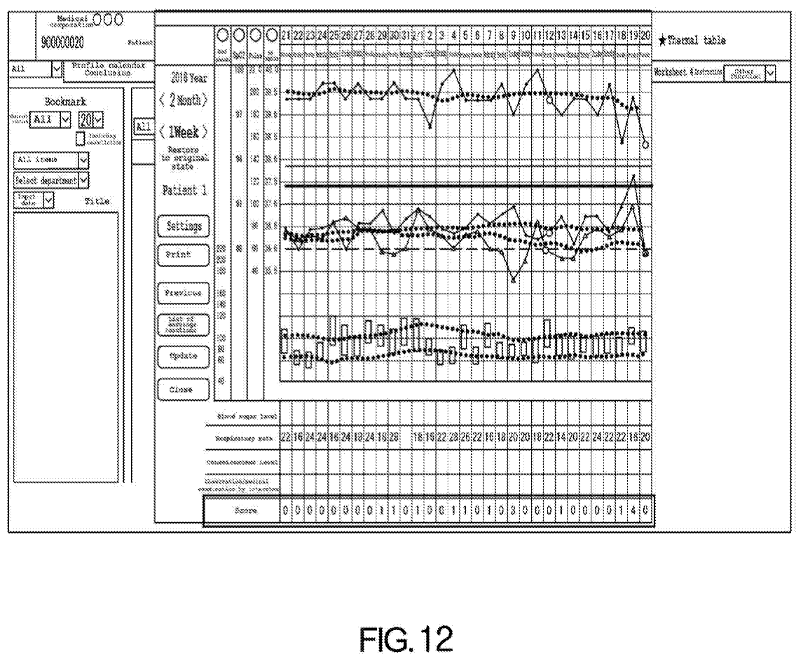

In addition, FIG. 12 shows an image showing the value of the score value information based on the content of the vital information in the column chart which is one of the display information of the electronic chart in the electronic chart used in the terminal installed in the hospital or the like. For example, the aspect of totaling a sum of the score values of several vital information and displaying the sum value of the score values for every day can be considered. In this case, the information based on the result of the scoring together with the information of the electronic chart in which the patient information is recorded can be used for the risk assessment of the subject.

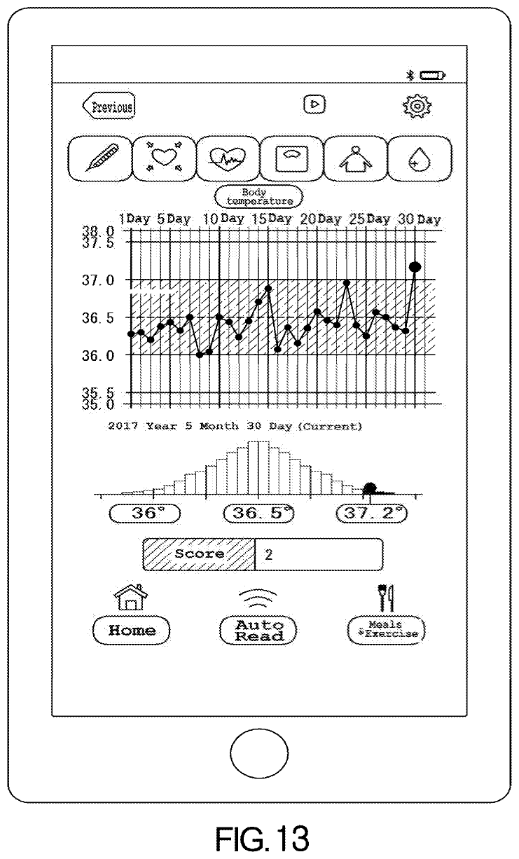

Further, FIG. 13 shows an image showing the value of the score value information based on the content of the vital information on the screen when the application software having the function of the software of the present invention is used in a smartphone terminal or the like. For example, there may be an aspect that indicates the recording (temperature) of vital information of the user of the smartphone terminal and the value of the score value information. In this case, the information based on the result of the scoring can be utilized for the evaluation of the state of health in the smartphone or the home health care.

[8. Determination of Measurement Accuracy with or without Normal Distribution and Determination of Abnormal Values]

In the health status determination device 1 to which the present disclosure is applied, the Q-Q plot can be used as a technique for checking whether the measured vital information is suitable for a normal distribution. For example, the vital standard deviation of the subject is plotted by taking the value of the vital standard deviation on the horizontal axis and the value of the percent point of the standard normal distribution corresponding to the cumulative probability of the standard deviation on the vertical axis. If each plot is located on a straight line, it is possible to visually confirm that the acquired vital information is normally distributed.

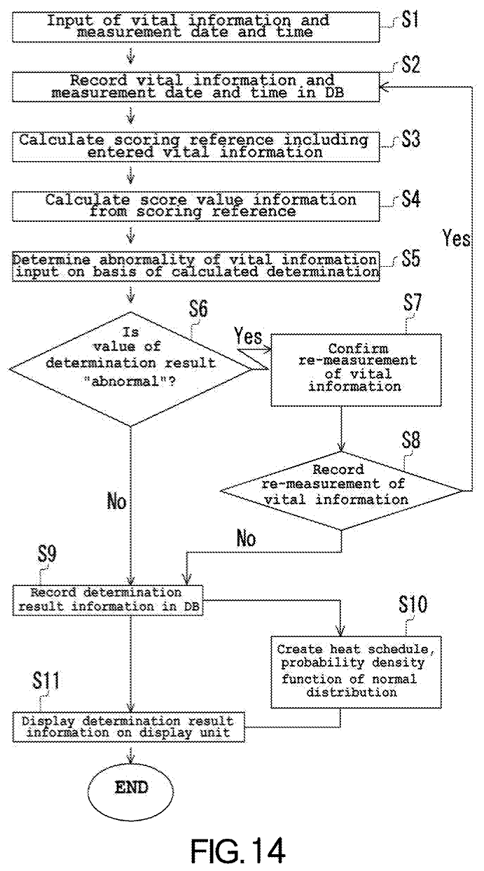

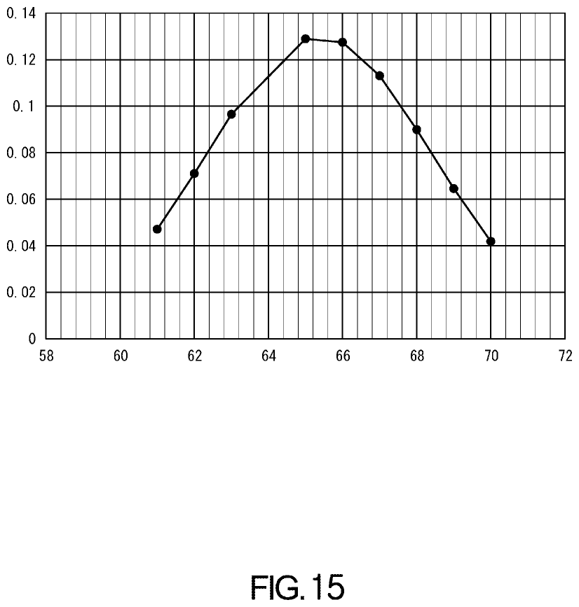

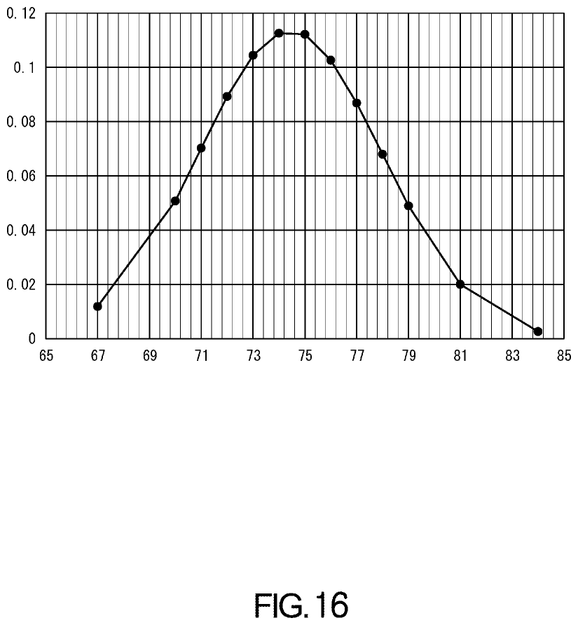

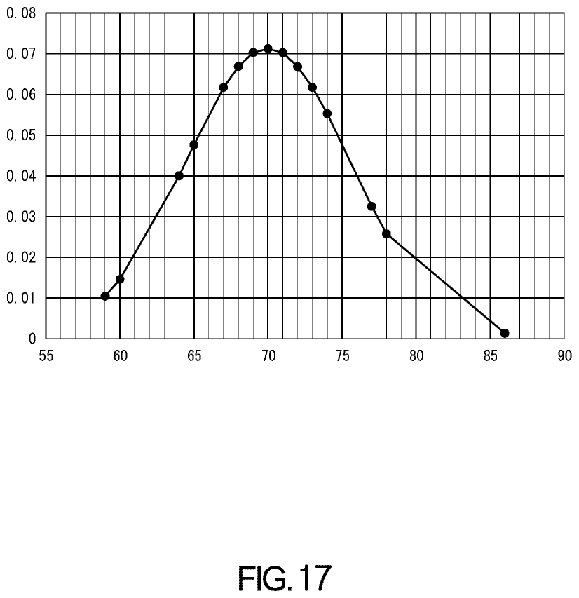

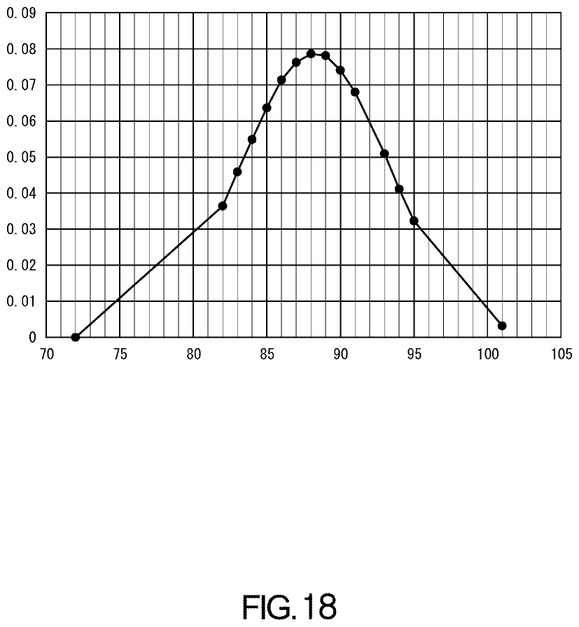

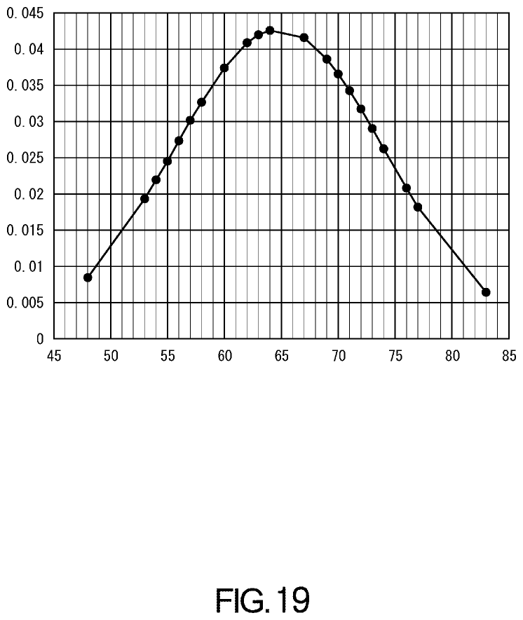

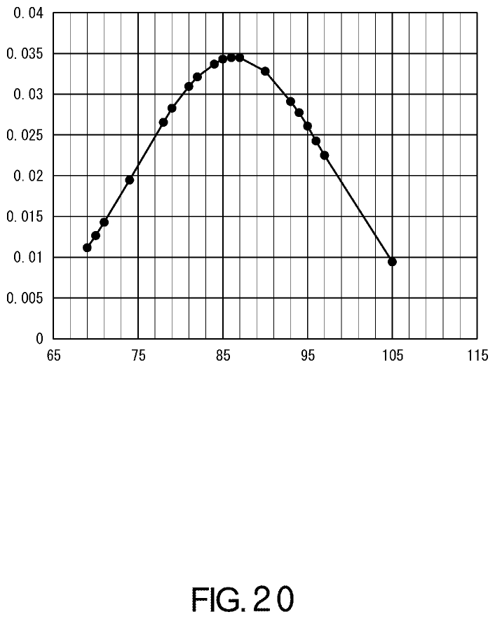

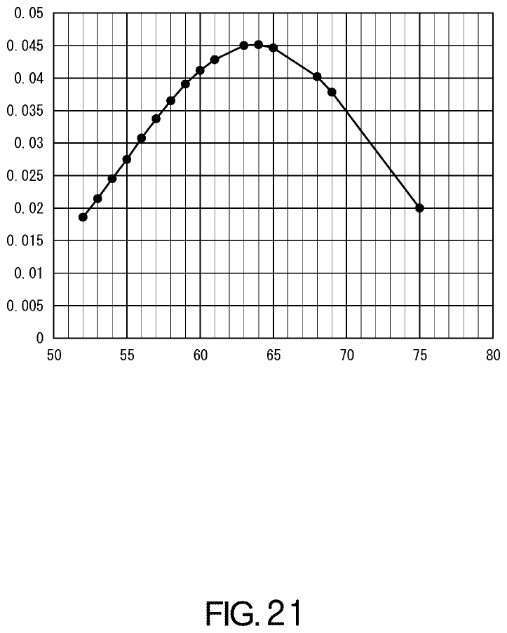

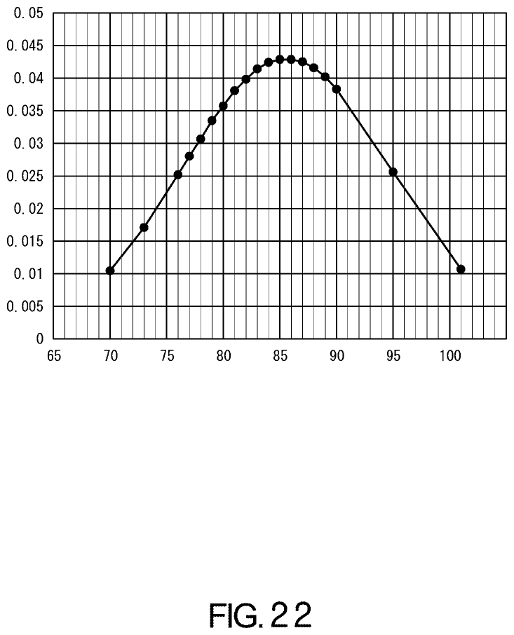

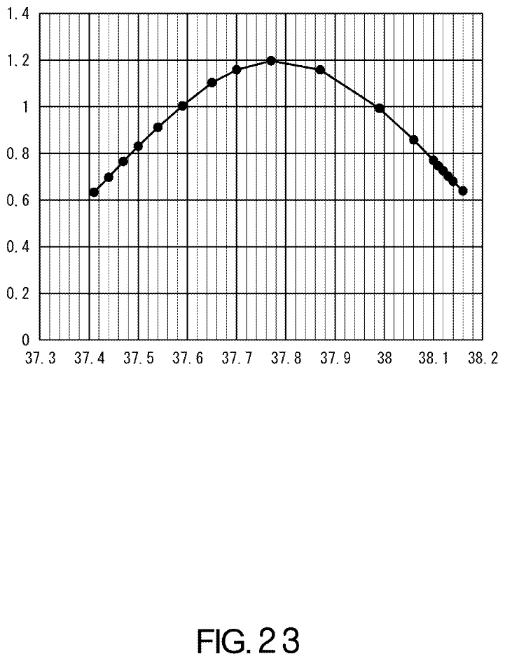

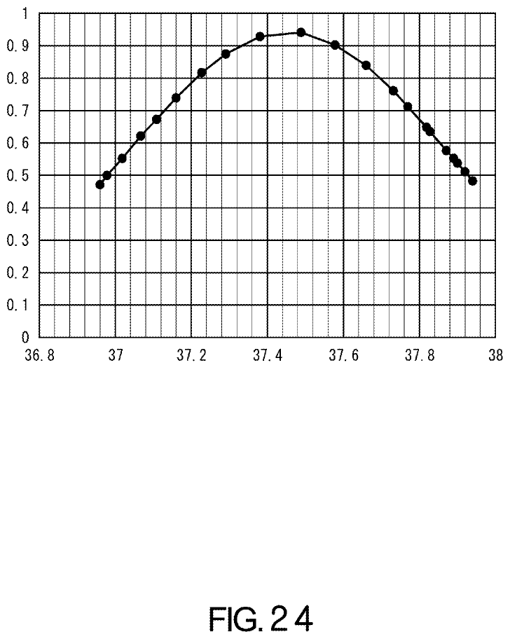

Next, a series of flows of information processing in the software to which the present invention is applied will be described with reference to FIG. 14.

FIG. 14 shows the flow of information processing from input of vital information to abnormality determination in score value information and display of resultant information.

First, the value of the subject's vital signs (temperature, pulse, systolic blood pressure, diastolic blood pressure, pulse pressure, oxygen saturation, and respiratory rate) is measured by each measuring device, and the measurement values and information on the date and time of measurement are inputted. (S1). At this time, the corresponding information of the observation information of the vital reference information 102 is selected or input from the evaluation result of the subject's consciousness level. The inputted information is recorded in the information recording section 4 (DB) as vital information of the subject (S2).

The calculation unit 2 functions as the reference calculation means 5, including vital information that is the subject of the determination recorded in the information recording section 4, to calculate the scoring reference information 102 (and to determine the vital decision reference information 102a). Calculation) (S3). Here, a vital mean value and a vital standard deviation are calculated, and based on these values, the scoring reference information (a predetermined numerical range, etc.) (and a predetermined numerical range for vital determination) under the set conditions are created. Here, the criteria relating to body temperature, pulse rate, systolic blood pressure, diastolic blood pressure, pulse pressure and respiratory rate are calculated at each time of scoring (and upon determination of the value of the vital signs).

Subsequently, the score value information 103 is calculated for each vital information by the scoring processing means 100 based on the scoring reference information 102 with respect to the vital information of the input target of determination.

When the score value information 103 is calculated, the determination processing means 6 determines whether the score value information is an abnormal value based on the determination criteria (S5). The determination result information is recorded in the information recording section 4 (DB) (S9), and the information of the determination result is displayed on the display section 3b as not being judged to be an “abnormal value (caution or warning)” as a result of the determination (S11). Further, based on the subject's vital information, a column chart (see FIG. 11) that graphs changes over time in the value of the vital sign and the established density function of the normal distribution (graph of the normal distribution curve) are created as display information (S10), and this information can also be confirmed on the display section 3b.

In addition, based on the reference of determination on the vital information of the target object of the input determination, in relation to the score value information determined as “the abnormal value (caution or warning)” as a result of the determination, for example, the display unit 3b may display the indication “Do you wish to perform re-measurement?” or recommend attention to the posture at the time of acquisition of the vitals, and then may confirm to the subject whether the re-measurement of vital information occurs (S7).

Here, when the subject or the caregiver selects “no re-measurement of vital information”, the determination result information with the abnormal determination is recorded in the information recording section 4 (DB) (S9), and the information of the determination result is displayed on the display section 3b.) is displayed (S11). Further, a thermal table (see FIG. 11) or an established density function of a normal distribution (graph of a normal distribution curve) is prepared as display information (S10), and this information can also be confirmed by the display unit 3b.

In addition, the information recording unit 4 records the value of the vital sign which is the result of determination with the abnormal determination so as to be included in the vital information 8. As a result, the vital information 8 accumulates both the vital information in which the score value information is determined to be a normal value and the vital information in which the score value information is determined to be an abnormal value. By collecting at least 30 pieces of information, it is possible to reflect the variation in the individual unique to the individual, create a normal distribution, and grasp the abnormal value of the vital sign in the target individual.

In addition, if the subject or the caregiver selects “Perform re-measurement of vital information”, it prompts the input of the value of the remeasured vital sign and the measurement date and time, and the information in which the inputted remeasurement vital information is input is remeasured by the subject. The information is recorded in the information recording unit 4 (DB) as re-measurement vital information of the subject (S2). After that, calculation (S3) of scoring reference information and abnormality determination (S4) of the score value information again are performed. In the determination, if the determination is not an abnormal value, the determination result information is recorded in the information recording section 4 (DB) (S9). In addition, when it is determined that it is an abnormal value, it may proceed to the step of confirmation of presence or absence of re-measurement vital information (S6), or it may progress to recording of determination result information (S9) as it is a 2nd determination result.

Although details are not shown in FIG. 14, the determination processing means 6 determines whether or not the value of the input vital sign is an abnormal value based on the vital decision reference information 102a. For the result of the determination, the determination result information is recorded in the information recording section 4 (DB), and the information of the determination result is displayed on the display section 3b as not being determined to be an “abnormal value (warning)”.

In addition, with respect to the input vital information of the subject of determination, when the value of the vital sign is determined to be an “abnormal value (warning)” as a result of the determination, based on the vital determination reference, for example, the display unit 3b shows, “Will you perform re-measurement?” or recommends attention to the posture at the time of the acquisition of the vital sign, and confirms to the subject whether there is re-measurement of vital information.

Here, when the subject or the caregiver selects “no re-measurement of vital information”, the determination result information of abnormal determination is recorded in the information recording unit 4 (DB), and the information of the determination result is displayed on the display unit 3b.

In addition, the information recording unit 4 records the value of the vital sign which has been determined as an abnormal determination to be included in the vital information 8. As a result, the vital information 8 accumulates both the vital information in which the score value information is determined to be a normal value and the vital information in which the score value information is determined to be an abnormal value. That is, not only the abnormality of the above-described score value information, but also the data of the abnormality of the value of the vital sign can be accumulated. By collecting at least 30 pieces of vital information, it is also possible to create a normal distribution in which the variation in the individual unique to the individual is reflected and to identify abnormal values of the vital signs in the subject individual.

When the subject confirms the information of the determination result on the display unit 3b, a series of information process is completed. In the above-described flow, the software to which the present disclosure is applied performs the determination of the health status from the vital information.

### INDUSTRIAL APPLICABILITY

Hereinafter, the further use case of the content of the health status determination device to which the present disclosure is applied will now be described.

[Interwork with Electronic Charts and Medical Systems]

The health status determination device to which the present disclosure is applied can be conceived in the aspect of interworking with an electronic chart introduced into a hospital. Since the information on the patient of the hospital where an electronic chart is installed is recorded in the electronic chart, by interworking with the information managed by the software to which the above-described disclosure is applied, more detailed information on a subject's condition related to the underlying disease, history, medication record, progress observation information, etc. can be used.

Further, since the information on the progress of the diagnosis by the doctor can be confirmed, the accuracy of the determination of the health status and the ease of use as a diagnosis support tool are improved. In addition, by recording the diagnosis result or the test result in the hospital in addition to the information of the subject, the amount of information on the individual is increased, resulting in a more accurate determination. In addition, it becomes more useful as a diagnostic support tool.

Moreover, an aspect which combines the health status determination device to which the present disclosure is applied, and the system of a remote image diagnosis can also be considered. For example, a device capable of acquiring image information such as a camera may be connected to a health status determination device, and image information of the subject may be transmitted to the hospital for remote diagnosis. At this time, the information of the subject's thermal table is also transmitted to the terminal on the hospital side, thereby enabling the doctor to diagnose remotely while confirming the abnormality of the subject's vital value.

Moreover, the health status determination device to which the present disclosure is applied may be subject to modification of several operation boards according to the level of a user. For example, in the aspect of cooperating with the above-mentioned electronic chart, it may become a diagnostic support tool at the time of doctor's diagnosis in a hospital. Here, the information managed by the diagnostic device accumulates the information of the daily diagnosis in the hospital, which can contribute to the improvement of the diagnosis level of the doctor.

In addition, by giving a specific nurse a tablet terminal having the function of the present invention in a hospital, it becomes possible to use it as an auxiliary tool when a nurse performs a diagnosis on behalf of a doctor. Furthermore, it is also helpful for nurses with tablet terminals to visit and care for the elderly at home or a facility.

Moreover, when the staff, such as a nursing facility, uses the device, it becomes possible to improve the diagnostic level for a specific subject by accumulating the information specialized for a resident.

[Application to Disease Prevention Technology]

It is conceivable to use a health status determination device to which the present disclosure is applied in a technique for preventing a disease of which onset can be predicted due to variations in vital signs such as brain disease, pneumonia, heart failure and dehydration. The above-mentioned diseases often cause fluctuations in the vital signs specific to the disease at the time of onset, and prevention of onset can be brought about by setting the fluctuations in the vital signs particular to the disease as the determination conditions.

[Usage in Personal Health Records (PHR) and Medical Big Data]

In recent years, the development of information and communication technology called ICT has been remarkable, and with the rise of the internet communication environment using information terminals such as a smart phones or wireless terminals and cheap and large databases such as cloud servers, large amounts of data can be transmitted and received at high speed. As a result, all data, so-called big data, are gathered from all over the world, and services for various purposes are achieved by using necessary data.

The Ministry of Health, Labor and Welfare of Japan intends to realize a “community comprehensive care system” that provides comprehensive medical, nursing, prevention, living, and living support for the elderly by 2025. However, most of the medical information on which it is based is isolated at each establishment.

For example, in information sharing among medical institutions, since it is still not rare to use facsimile or continuity of health data among facilities, compared to the era of the Fourth Industrial Revolution, it is showing an aspect of a pre-era. Establishment of a medical information alliance network is being attempted in more than 170 locations around the country, but it has not been able to cover the entire region and population, and has not been integrated into a common platform due to a problem of operating costs or a problem of low utilization rates.

The Japanese Ministry of Health, Labor and Welfare is considering the PHR initiative with the aim that “Each citizen manages health, medical, and care information that spans his or her entire life, and utilizes that information to receive good quality services that are consistent with his or her health status” (from the “Ministry of Health and Welfare Labor,” A Meeting on the Direction of Medical ICT in the Cloud Era”). PHR is an abbreviation of Personal Health Records, and it is a structure that enables an individual to collect or preserve medical information or health information about himself and to utilize it throughout his life.

According to this, “it is necessary to refer not only to the latest information but also to the past information, so that you can manage the information of your whole life and use it for the service you want to use and when you want to use it.” When this PHR initiative is realized, even if there is no platform integration, various operations are possible through medical treatment, caring, health, and personal information cooperation such as “digitization of the nursing prevention handbook”, “electronification of medical examination/examination information in municipalities or workplaces, and management of vital data/life-related information”.

In addition, along with the “PHR initiative”, various department are cooperating to make it easy to select the necessary data by unifying the data format (API, etc.) to create an environment where big data can be easily used, and a “medical ID” scheme, which makes it easier to continuously identify personal data, called a personal medical number, is also planned for the “Amendment of Personal Information Protection Act,” which allows anonymous information to be used without permission from individuals for development or treatment.

This initiative addresses vital data as follows: “The so-called secondary use, which anonymizes the person's health, medical and care information, and then analyzes and uses it as big data, should be considered as an important factor in reviewing the sustainability of PHR services. Particularly, for vital data, various devices and services exist in the market, but when integrating with PHR and utilizing data, considering the secondary use for the purpose of analyzing the effects of health care, it is desirable to unify APIs between platforms that collect PHRs.”

In the future, an aspect of interworking health status determination devices (or the health status determination method) to which the present disclosure is applied may be considered. When integrating this device to utilize vital data, the major difference from the existing technology is that when performing health care, “Tailor-made Diagnosis” or so-called “Personalized Medical Care”, which is tailored to the characteristics of each person, is not compared with general data. Furthermore, the interpretation of medical big data opens the way for preventive medicine and helps “artificial intelligence” to support it. In the future, it is considered that the health status determination device to which the present invention is applied will greatly contribute to the realization of the PHR concept.

[Doctor, Nurse Training Software]

Moreover, the health status determination device of the present disclosure can also be used as an educational tool of a medical worker. In addition to the present disclosure, it is possible to create educational software that combines a questionnaire and an answer based on this information by combining a conditional discrimination flowchart database. In addition, scores of a plurality of medical practitioners using educational software may be recorded and ranked to prepare a doctor or nurse evaluation table.

[Reference Tool when Nurse Prescribes Medicine]

It may also be considered for use as a reference tool when nurses prescribe medication. For example, information on the subject's medication history is recorded in the subject's personal information. Thereby, the data on “what kind of medicine was prescribed at the time of a symptom” is accumulated, and a pharmacist can utilize this as reference information at the time of medication. Moreover, depending on the kind of medicine, it can be extended also to the use for prescription, without going through a pharmacist. Checking the medication history by the pharmacist can also be easily performed.

[Drug Management and Delivery Service]

Furthermore, by linking the medication history record and delivery service, it is possible to automatically obtain the medicine that a subject regularly needs when necessary.

[Link with Medical Examination Data at Work and School]

It is also conceivable that the health status determination device of the present invention records and utilizes information of periodic medical examinations at work or school. In this case, the period for acquiring the vital information is shortened, so the caution, warning, and abnormality determination are appropriately configured. This can be helpful for the health care of the subject. It is also a means of acquiring vast clinical data. Furthermore, comprehensive health care of the subject becomes possible by linking with the information of the medical examination which a public institution performs.

[Health Care in the Remote Area]

The health status determination device of the present invention can also be used for health care at a remote site. For example, the health determination device of the present invention may be installed in the country of origin, a sea-going fishing ship, or an overseas base of an army. This makes it possible to manage the health of the subject even in countries with low medical standards or in places where no medical facilities exist. In addition, the diagnosis by a doctor can also be performed by combining with the remote diagnosis equipment as described above.

[Check the Disease Outbreak by Region]

The health status determination device of the present invention can contribute to the prophylactic medical care of local medical care by linking it with information on the disease occurrence status for each region. For example, linking with information about the prevalence of influenza can lead to preventive measures in areas where health condition determination devices are used. In addition, the subject's information in the epidemic area can be utilized as clinical data.

[Detection of Air Environment]

Furthermore, the detection mechanism of an air environment and the health status determination device of this invention can also be combined. The concentration of formaldehyde or PM 2.5 can be detected by the air environment detecting mechanism, and the air pollution level in the area can be determined from the concentration to prompt awareness to the user of the device. In addition, it can be used as an information acquisition tool for alerting the residents of the target area or improving the environment in conjunction with administrative services.

[Utilization for Adjustment of Indoor Environment]

Based on the information recorded in the state-of-health determination device of the present invention, the use in a healthcare facility that enables the adjustment of room temperature and humidity by an air conditioner can be considered. It is possible to control the environment to be a room temperature or humidity suitable for the subject whose vital sign values are determined to be an abnormal value, or to control an appropriate temperature at which it is easy to maintain a healthy state.

[Care Recording Software and Care Request Software]

The state of health determination device to which the present invention is applied can be predicted to be linked to the care recording software or the care request software. The information management unit manages the information of the nursing record input to the nursing recording software to accumulate data on “what kind of nursing is appropriate and for what symptoms.” As a result, it is possible for the caregiver to provide uniform services to the caregiver without depending on the function level of the caregiver. Another aspect in conjunction with care recording software is described below.

In the case of linking with the care request software, it can be used as a support tool for calculating the care costs. This makes it possible to easily check the costs incurred for the provided care contents, which can lead to an improvement in work efficiency.

[Health Check of Care Staff]

The health determination device to which the present invention is applied can also be utilized for checking the state of health of the caregiver. Health care is performed by measuring vital information of the caregiver employee and transmitting it to the health status determination device. This can bring about an improvement in the working environment at the care site.

[Care Function]

In addition, in the case of linking with a nursing facility or a care system for the elderly of a single household, in the case where abnormality of the vital value is determined or abnormality is observed in the operation of the subject, a configuration may be considered in which notification is given to the relative of the care subject (for example, family). For example, by installing a person sensor in the house, an alarm is automatically sent to the security company or family when the owner does not move for a while in the bathroom. At this time, the recording of the vital information of the subject to be observed may be simultaneously transmitted as data.

[Diet, Condition Management]

The health status determination device to which the present invention is applied can also be utilized as a device for supporting diet and condition management of a user. For example, a configuration may be adopted in which advice for weight loss is displayed based on vital information and information on calories ingested in a meal. It is also possible to provide a plurality of weight loss programs in cooperation with facilities such as training gyms.

[Utilization in Wearable Device]

The structure which links the health status determination device of this invention with a wearable device is also conceivable. In recent years, a small wearable device that can be attached to a body has been developed, and it is possible to acquire various vital information such as body temperature, pulse rate, systolic blood pressure, and diastolic blood pressure in real time using such a device. In the health status determination device of the present invention, the application range can be greatly broadened by combining it with the wearable device as the display means for acquiring the vital information and the determination result. In addition, the health status determination device can be utilized as a device for self-management to perform condition management on its own.

[Utilization of Application Software]

In addition, as mentioned above, the structure which makes it possible to use the function of the health status determination device of this invention as application software, and to make it available to a portable terminal or a tablet terminal can be considered. As a result, the functions of the device can be used easily, thereby improving convenience. In addition, it is possible to contribute to the improvement of the diffusion rate of the device, resulting in the acquisition of wider clinical data.

[Shopping Support Software]

It is also conceivable to link the information recorded in the health status determination device of the present invention with a product sales website on the Internet or software that supports product purchase. By providing a function of recommending a food, a health device, or the like according to the state of health of the user, reference information when purchasing a product can be obtained.

[Animal Health Care]

The health status determination device of the present invention is also contemplated for use in animals. In addition to humans, it can contribute to the health care of pets, zoo animals, and wildlife. In addition, by accumulating the clinical data and diagnostic information of the animal, medical and scientifically useful information can be obtained.

[Installation in a Vehicle]

The aspect which installs the health status determination device of this invention in a vehicle can be adopted. For example, a vital instrument (e.g., a thermometer, pulse meter, respiratory rate sensor, etc.) may be installed in the driver's seat, and a warning may be given if it is suspected that a driver's condition is poor. In addition, the alcohol detector can be configured to perform a check for drunk driving.

As described above, the software of the present invention is capable of grasping the variation in individual subjects with high accuracy by reflecting the vital signs and the daily conditions in consideration of the individual differences of the subjects. Therefore, it contributes to the health care of the target person and the provision of medical care suited to the characteristics of each person.

In addition, the health status determining device of the present invention can grasp the variation in individual subjects with high accuracy by reflecting the vital signs and the daily conditions in consideration of the individual differences of the subjects. Therefore, it contributes to the health care of the target person and the provision of medical care suited to the characteristics of each person.

In addition, according to the health status determination method of the present invention, it is possible to grasp the variation in individual subjects with high accuracy by reflecting the vital signs or daily conditions in consideration of the individual differences of the subjects. Therefore, it contributes to the health care of the target person and the provision of medical care suited to the characteristics of each person.

### LIST OF REFERENCE CHARACTERS

1 Health status determination device
1a Software
2 Calculating unit
2a Calculating unit
3 Tablet terminal
3a Input unit (of the tablet terminal)
3b Display unit (of the tablet terminal)
3c Information transmission/reception unit (of the tablet terminal)
4 Information recording unit
4a Information recording unit
5 Reference calculation means
5a Reference calculation means
6 Determination processing means
6a Determination processing means
7 Personal information
8 Vital information
9 Reference time information
10 Posture information
11 Temperature information
12 Score determination result information
13 Re-measured vital information
14 Mean calculation means
15 Standard deviation calculation means
16 Normal distribution calculation means
18 Score determination reference information
21a Vital meter
21b Thermometer
22a Smart phone terminal
22b Personal computer terminal (PC terminal)
23 Information input means
24 Information recording means
24a Information recording means
30a Internet
32a Information management server
32b Software
32c Software
32d Software
50a User terminal
50b External terminal
60a User terminal
60b External terminal
70b Management terminal
100 Scoring processing means
100a Scoring processing means
101 Scoring reference configuring means
102 Scoring reference information
110 Mode calculation means

1. A system for scoring vital information, the system comprising:
one or more programs configured to score vital information related to obtained vital signs to determine a health status of a subject based on an obtained score result information;
at least one processing unit to execute the one or more programs;
a storage unit to store the one or more programs; and
an input unit,
wherein the one or more programs, when executed, causes an information processing device to function as:
an information input means for receiving an input of the vital information obtained from the subject wherein the input of the vital information including further includes at least one measurement value selected from classical vital signs according to a first normal distribution, and an acquisition date and time information;
an information recording means for recording the input of the vital information and the acquisition data and time information as recorded vital information;
a reference calculation means for calculating a mean μ and a standard deviation σ of all or a part of a plurality of the recorded vital information;
a scoring processing means for scoring the input of the vital information to calculate score result information wherein the score result information includes a value of a score, based on a predetermined scoring condition and wherein the predetermined scoring condition is based on a second normal distribution having at least the mean μ as a peak value; and
a score determination means for determining whether the score result information is a first abnormal value, based on a predetermined score determination condition,
wherein the vital information includes a value determined by the score determination means as a second abnormal value,
wherein the vital information of at least 30 pieces of measurement data reflects variation unique to the subject,
wherein the first normal distribution is made up of the vital information of at least 30 pieces of measurement data,
wherein the vital information further includes at least one measurement value selected from body temperature, blood pressure, pulse rate and pulse pressure which are the classic vital signs, and
wherein a scoring condition, for the at least one measurement value selected from body temperature, blood pressure, pulse rate and pulse pressure, has the value of Equation 1 represented below using the mean μ, the standard deviation σ, n, and m that are greater than zero (0) as a lower limit, a value of Equation 2 below as an upper limit, and at least one of the lower limit and the upper limit as a reference:
μ−nσ Equation 1; and
μ+mσ Equation 2.

2. A system for scoring vital information, the system comprising:
one or more programs configured to score vital information related to obtained vital signs to determine a health status of a subject based on an obtained score result information;
at least one processing unit to execute the one or more programs;
a storage unit to store the one or more programs; and
an input unit,
wherein the one or more programs, when executed, causes an information processing device to function as:
an information input means for receiving an input of the vital information obtained from the subject, wherein the input of the vital information includes at least one measurement value selected from classical vital signs according to a first normal distribution and an acquisition date and time information;
an information recording means for recording the input of the vital information and the acquisition data and time information as recorded vital information;
a reference calculation means for calculating a mean μ and a standard deviation σ of all or a part of a plurality of the recorded vital information;
a scoring processing means for scoring the input of the vital information to calculate score result information wherein the score result information includes a value of a score, based on a predetermined scoring condition and wherein the predetermined scoring condition is based on a second normal distribution having at least the mean μ as a peak value;
a score determination means for determining whether the score result information is a first abnormal value, based on a predetermined score determination condition, and
a vital determination means for determining whether an input predetermined vital information is a second abnormal value based on a predetermined numerical range for vital determination wherein the predetermined numerical range for vital determination is based on at least one selected from the mean μ and the standard deviation σ, and the predetermined numerical range for vital determination is based on a third normal distribution having at least the mean μ as a peak value,
wherein the vital information comprises:
a value determined by the score determination means as a third abnormal value; and
at least one measurement value selected from body temperature, blood pressure, pulse rate and pulse pressure,
wherein the vital information of at least 30 pieces of measurement data reflects variation in the subject unique to the subject,
wherein the first normal distribution is made up of the vital information of at least 30 pieces of measurement data, and
wherein the vital determination means has a value of Equation 1 represented below using the mean μ, the standard deviation σ, n, and m that are numbers greater than zero (0) as a lower limit, and a value of Equation 2 below as an upper limit, and the vital determination means determine whether the input predetermined vital information is a fourth abnormal value based on at least one of the lower limit and the upper limit:
μ−nσ Equation 1; and
μ+mσ Equation 2.

3. The system for scoring vital information according to claim 1,
wherein the vital information further includes:
at least one measurement value selected from body temperature, blood pressure, pulse rate and pulse pressure which are the classic vital signs;
a measurement value of oxygen saturation degree;
a measurement value of respiratory rate; and
a result of consciousness level evaluation obtained by observing the consciousness level,
wherein the scoring condition includes:
a first predetermined numerical range for the at least one measurement value selected from body temperature, blood pressure, pulse rate and pulse pressure based on a third normal distribution using the mean μ as a peak value,
a second predetermined numerical range for the measurement value of oxygen saturation degree,
a third predetermined numerical range based on a mode or the mean μ of all or part of recorded values of the respiratory rate from the measurement value of the respiratory rate; and
a predetermined observation state indicating a degree of consciousness level, for a consciousness level evaluation result.

4. The system for scoring vital information according to claim 1,
wherein the score determination means determines whether the score is a third abnormal value with respect to a total point of the score result information that is scored from the vital information.

5. The system for scoring vital information according to claim 1,
wherein the information input means receives input of information on a disease status, information on a history, observation information on a condition, and information on a care record in of the subject, and
the information recording means records the information on the disease status, the information on the history, the observation information on the condition, and the information on the care record of the subject,
wherein the one or more programs further causes the information processing device to function as a thermal table processing means for creating a thermal table based on the vital information recorded in the information recording means, information on a hospital bed, the information on the history, the observation information on the condition, and information on the vital information from the care record of the subject.

6. The system for scoring vital information according to claim 1, wherein the reference calculation means calculates the mean μ and the standard deviation σ from at least two pieces of the vital information of a predetermined period recorded in the information recording means.

7. The system for scoring vital information according to claim 1, wherein the score judging means determines an abnormality by dividing the abnormality into at least two stages when the score judging means determines the score result information as a third abnormal value.

8. The system for scoring vital information according to claim 2, wherein a scoring condition, for the at least one measurement value selected from the body temperature, the blood pressure, the pulse, and the pulse pressure, has a value of Equation 1 represented using the mean μ, the standard deviation σ, n and m that are numbers greater than zero (0) as a lower limit, a value of Equation 2 as an upper limit, and at least one of the low limit and the upper limit as a reference.

9. A health status determination device for scoring vital information related to obtained vital signs to determine a health status of a subject based on an obtained score result information, the device comprising:
an information input means for receiving an input of the vital information obtained from the subject, wherein the input of the vital information further includes at least one measurement value selected from classical vital signs according to a first normal distribution, and an acquisition date and time information;
an information recording means for recording the input of the vital information and the acquisition data and time information as recorded vital information;
a reference calculation means for calculating a mean μ and a standard deviation σ of all or part of a plurality of the recorded vital information;
a scoring processing means for scoring the input of the vital information to calculate score result information wherein the score result information includes a value of a score, based on a predetermined scoring condition and wherein the predetermined scoring condition is based on a second normal distribution having at least the mean μ as a peak value;
a score determination means for determining whether the score result information is a first abnormal value, based on a predetermined score determination condition; and
a display means capable of displaying a determination result determined by the score determination means,
wherein the vital information includes a value determined by the score determination means as a second abnormal value,
wherein the vital information of at least 30 pieces of measurement data reflects variation unique to the subject,
wherein the first normal distribution is made up of the vital information of at least 30 pieces of measurement data,
wherein the vital information further includes at least one measurement value selected from body temperature, blood pressure, pulse rate and pulse pressure which are the classic vital signs, and
wherein the scoring condition, for the at least one measurement value selected from the body temperature, the blood pressure, the pulse, and the pulse pressure, has a value of Equation 1 represented below using the mean μ, the standard deviation σ, n and m that are numbers greater than zero (0) as a lower limit, a value of Equation 2 below as an upper limit, and at least one of the low limit and the upper limit as a reference:
μ−nσ Equation 1; and
μ+mσ Equation 2.

10. A health status determination device for scoring vital information, related to obtained vital signs to determine a health status of a subject based on an obtained score result information, the device comprising:
an information input means for receiving an input of the vital information obtained from the subject, wherein the input of the vital information further includes at least one measurement value selected from classical vital signs according to a first normal distribution, and an acquisition date and time information;
an information recording means for recording the input of the vital information and the acquisition data and time information as recorded vital information;
a reference calculation means for calculating a mean μ and a standard deviation σ of all or a part of a plurality of the recorded vital information;
a scoring processing means for scoring the input of the vital information to calculate score result information wherein the score result information includes a value of a score, based on a predetermined scoring condition and wherein the predetermined scoring condition is based on a second normal distribution having at least the mean μ as a peak value;
a score determination means for determining whether the score result information is a first abnormal value, based on a predetermined score determination condition;
a vital determination means for determining whether an input predetermined vital information is a second abnormal value, the second abnormal value based on a predetermined numerical range for vital determination wherein the predetermined numerical range for vital determination is based on at least one selected from the mean μ and the standard deviation σ, and the predetermined numerical range for vital determination is configured based on a third normal distribution having at least the mean μ as a peak value; and
a display means capable of displaying a determination result determined by the score determination means,
wherein the vital information comprises:
a value determined by the vital determination means as an abnormal value and the score determination means performs the next determination; and
at least one measurement value selected from body temperature, blood pressure, pulse rate and pulse pressure which are the classic vital signs,
wherein the vital information of at least 30 pieces of measurement data reflects variation unique to the subject,
wherein the first normal distribution is made up of the vital information of at least 30 pieces of measurement data,
and
wherein the vital determination means has a value of Equation 1 represented below using the mean μ, the standard deviation σ, n, and m that are numbers greater than zero (0) as a lower limit, and a value of Equation 2 below as an upper limit, and determines whether the input predetermined vital information is a fourth abnormal value, based on at least one of the lower limit and the upper limit:
μ−nσ Equation 1; and
μ+mσ Equation 2.

11. A health status determination method, executed by a computer, for scoring vital information related to obtained vital signs to determine a health status of a subject based on an obtained score result information, the method comprising:
an information recording process for receiving an input of the vital information obtained from the subject wherein the vital information includes at least one measurement value selected from classical vital signs according to a first normal distribution;
a reference calculating process for calculating a mean μ and a standard deviation σ of all or a part of a plurality of the vital information recorded from the information recording process;
a scoring processing process for scoring the vital information inputted during the information recording process to calculate score result information wherein the score result information includes a value of a score based on a predetermined scoring condition and wherein the predetermined scoring condition is based on a second normal distribution having at least the mean μ as a peak value; and
a score determination process for determining whether the score result information is a first abnormal value based on a predetermined score determination condition,
wherein the vital information comprises:
a value determined by the score determination process as a second abnormal value, and the score determination process performs next determination; and
at least one measurement value selected from body temperature, blood pressure, pulse rate and pulse pressure which are the classic vital signs,
wherein the vital information of at least 30 pieces of measurement data reflects variation in the individual unique to the individual,
wherein the first normal distribution is made up of the vital information of at least 30 pieces of measurement data,
and
wherein the scoring condition, for at least one measurement value selected from the body temperature, the blood pressure, the pulse rate and the pulse pressure, the value of Equation 1 displayed below using the mean μ and the standard deviation σ, a number larger than 0 and m is a lower limit, and a value of Equation 2 below is an upper limit, and at least one of the lower limit and the upper limit is configured as a reference:
μ−nσ Equation 1; and
μ+mσ Equation 2.

12. A health status determination method, executed by a computer, for scoring vital information related to obtained vital signs to determine a health status of a subject based on an obtained score result information, the method comprising:
an information recording process for receiving an input of the vital information obtained from the subject wherein the vital information includes at least one measurement value selected from classical vital signs according to a first normal distribution;
a reference calculating process for calculating a mean μ and a standard deviation σ of all or a part of a plurality of the vital information recorded from the information recording process;
a scoring processing process for scoring the vital information inputted during the information recording process to calculate score result information wherein the score result information includes a value of a score based on a predetermined scoring condition and wherein the predetermined scoring condition is based on a second normal distribution having at least the mean μ as a peak value;
a score determining process for determining whether the score result information is a first abnormal value based on a predetermined score determination condition; and
a vital determination process for determining whether an input predetermined vital information is a second abnormal value based on a predetermined numerical range for vital determination wherein the predetermined numerical range for vital information is based on at least one selected from the mean μ and the standard deviation σ, and the predetermined numerical range for vital determination is based on a third normal distribution having at least the mean μ as a peak value;
wherein the vital information comprises:
a value determined by the vital determination process as a third abnormal value; and
at least one measurement value selected from body temperature, blood pressure, pulse rate and pulse pressure,
wherein the vital information of at least 30 pieces of measurement data reflects variation unique to the subject,
wherein the first normal distribution is made up of the vital information of at least 30 pieces of measurement data, and
wherein the vital determination process has a value of Equation 1 represented below using the mean μ, the standard deviation σ, n and m that are numbers greater than zero (0) as a lower limit and a value of Equation 2 below as an upper limit, and the vital determination means determines whether the input predetermined vital information is a fourth abnormal value, based on at least one of the lower limit and the upper limit:
μ−nσ Equation 1; and
μ+mσ Equation 2.
